(19) United States
(12) Patent Application Publication
(10) Pub. No.: US 2022/0249659 A1
(43) Pub. Date: Aug. 11, 2022

(54) COMBINATION OF PD-1 INHIBITORS AND LAG-3 INHIBITORS FOR ENHANCED EFFICACY IN TREATING CANCER

(71) Applicant: Regeneron Pharmaceuticals, Inc., TARRYTOWN, NY (US)

(72) Inventors: Glenn KROOG, New York, NY (US); Tasha N. SIMS, New York, NY (US)

(21) Appl. No.: 17/611,073

(22) PCT Filed: May 12, 2020
(86) PCT No.: PCT/US2020/032515

§ 371 Date: Nov. 12, 2021

Related U.S. Application Data
(60) Provisional application No. 62/847,068, filed on May 13, 2019.

(51) Int. Cl. A61K 39/395; C07K 16/28; A61K 39/04; A61K 38/19; A61K 38/20; A61P 35/00; C07K 16/30

(52) U.S. Cl. CPC A61K 39/3955; C07K 2317/31; C07K 16/2803; A61K 39/04; A61K 38/193; A61K 38/2013; A61K 38/2046; A61K 38/20; A61K 38/208; A61K 38/2086; A61P 35/00; C07K 16/2809; C07K 16/2887; C07K 16/3092; C07K 2317/76; C07K 2317/21; C07K 16/2818

(57) ABSTRACT

The present disclosure provides methods for treating or inhibiting the growth of cancer comprising selecting a patient with cancer and administering a therapeutically effective amount of a LAG-3 inhibitor in combination with a therapeutically effective amount of a PD-1 inhibitor (e.g., an anti-PD-1 antibody or antigen-binding fragment thereof). In certain embodiments, the administration of the PD-1 inhibitor enhances the efficacy of a LAG-3 inhibitor (e.g., an anti-LAG-3 antibody or antigen-binding fragment thereof) in inhibiting the growth of cancer.

### TECHNICAL FIELD

The present disclosure provides, in part, compositions, including inhibitors of LAG-3 and PD-1, and methods for treating cancer.

### SEQUENCE LISTING

An official copy of the sequence listing is submitted concurrently with the specification electronically via EFS-Web as an ASCII formatted sequence listing with a file name of 10568WO01_SEQ_LIST_ST25, a creation date of May 12, 2020, and a size of about 20 kilobytes. The sequence listing contained in this ASCII formatted document is part of the specification and is herein incorporated by reference in its entirety.

### BACKGROUND

Programmed death-1 (PD-1) receptor signaling in the tumor microenvironment plays a key role in allowing tumor cells to escape immune surveillance by the host immune system. The PD-1 receptor has two ligands, PD-ligand-1 (PD-L1) and PD-L2. Blockade of the PD-1 signaling pathway has demonstrated clinical activity in patients with multiple tumor types, and antibody therapeutics that block PD-1/PDL1 signaling (e.g., nivolumab, pembrolizumab, atezolizumab, durvalumab, and cemiplimab) have been approved for the treatment of various cancers including, for example, metastatic melanoma and metastatic squamous non-small cell lung cancer.

Like PD-1, lymphocyte activation gene-3 (LAG-3) negatively regulates T-cell activity. LAG-3 (also called CD223) is a 503 amino acid transmembrane protein receptor expressed on activated CD4 and CD8 T cells, γδ T cells, natural killer T cells, B-cells, natural killer cells, plasmacytoid dendritic cells and regulatory T cells. LAG-3 is a member of the immunoglobulin (Ig) superfamily. The primary function of LAG-3 is to attenuate the immune response. LAG-3 binding to MHC class II molecules results in delivery of a negative signal to LAG-3-expressing cells and down-regulates antigen-dependent CD4 and CD8 T cell responses. LAG-3 negatively regulates the ability of T cells to proliferate, produce cytokines and lyse target cells, termed as ‘exhaustion’ of T cells. LAG-3 is also reported to play a role in enhancing T regulatory (Treg) cell function (Pardoll 2012, Nature Reviews Cancer 12: 252-264).

Since both PD-1 and LAG-3 play important roles in tumor immunity, they are ideal targets for immunotherapy. Targeting both LAG-3 and PD-1 (including in anti-PD-1 resistant tumors) may result in objective responses in patients across several tumor types.

### SUMMARY

The present disclosure relates to methods for treating cancer and methods for inhibiting tumor growth.

Provided herein are methods for treating, ameliorating at least one symptom or indication, or inhibiting the growth of cancer in a subject. The methods according to this aspect of the disclosure comprise administering a therapeutically effective amount of an antibody or antigen-binding fragment thereof that specifically binds to programmed death 1 (PD-1) in combination with a therapeutically effective amount of an antibody or antigen-binding fragment thereof that specifically binds to LAG-3 to a subject in need thereof.

In certain embodiments, methods are provided for treating cancer or inhibiting the growth of a tumor in a subject in need thereof. The methods according to this aspect of the disclosure comprise administering to the subject a therapeutically effective amount each of (a) an antibody or antigen-binding fragment thereof that specifically binds programmed death 1 (PD-1); and (b) an antibody or antigen-binding fragment thereof that specifically binds lymphocyte activation gene-3 (LAG-3). In some aspects, one or more doses of the anti-LAG-3 antibody are administered in combination with one or more doses of the anti-PD-1 antibody.

In some aspects, the treatment produces a therapeutic effect selected from the group consisting of delay in tumor growth, reduction in tumor cell number, tumor regression, increase in survival, partial response, and complete response. In some aspects, tumor growth is delayed by at least 10 days as compared to an untreated subject. In some aspects, tumor growth is inhibited by at least 50% as compared to an untreated subject. In some aspects, tumor growth is inhibited by at least 20% as compared to a subject administered with either antibody as monotherapy.

In some aspects, the inhibition is more efficacious than administration of either antibody as a monotherapy.

In certain embodiments, methods are provided for treating, ameliorating at least one symptom or indication, or inhibiting the growth of cancer in a subject. In certain embodiments, methods are provided for delaying the growth of a tumor or preventing tumor recurrence. The methods, according to this aspect, comprise sequentially administering one or more doses of a therapeutically effective amount of an antibody or antigen-binding fragment thereof that specifically binds to PD-1 in combination with one or more doses of a therapeutically effective amount of an antibody or antigen-binding fragment thereof that specifically binds to LAG-3 to a subject in need thereof.

in one embodiment, the anti-PD-1 antibody and anti-LAG-3 antibody are administered as a first (“front”) line of treatment (e.g., the initial or first treatment). In another embodiment, the anti-PD-1 antibody and anti-LAG-3 antibody are administered as a second line of treatment (e.g., after initial treatment with the same or a different therapeutic, including after relapse and/or where the first treatment has failed).

In certain embodiments, methods are provided for treating cancer or inhibiting the growth of a tumor. The methods, according to this aspect, comprise: (1) selecting a patient with a tumor, wherein the selected patient has received prior treatment comprising a PD-1 inhibitor or PD-L1 inhibitor; and (2) administering to the patient (a) 350 mg anti-PD-1 antibody comprising the HCVR/LCVR amino acid sequence pair of SEQ ID NOs: 1/2; and (b) 1, 3, 10, 20, or 40 mg/kg or 1600 mg anti-LAG-3 antibody comprising the HCVR/LCVR amino acid sequence pair of SEQ ID NOs: 11/12. In some aspects, the administering of step (2) occurs once every 3 weeks, or once every 6 weeks. In some aspects, the selecting of step (1) further identifies a patient as satisfying one or more of the following criteria: (i) ineligible for platinum based therapy, or tumor progression or recurrence within 6 months of last dose of platinum therapy; (ii) confirmed diagnosis of malignancy; (iii) demonstrated progression of a tumor for which there is no available therapy likely to convey clinical benefit; (iv) disease progression/recurrence after one platinum-containing regimen; (v) anti-PD-1/PD-L1 experienced stage IIIB, IIIC, or IV NSCLC with no more than 2 prior therapies for metastatic disease; (vi) anti-PD-1/PD-L1 experienced advanced or metastatic ccRCC with a clear cell component who had received no more than 2 previous regimens of anti-angiogenic therapy; (vii) anti-PD-1/PD-L1 experienced advanced or metastatic non-uveal melanoma who have received no more than 2 previous regimens for metastatic disease; (xiii) anti-PD-1/PD-L1 experienced relapsed/refractory DLBCL who have either progressed after or are not candidates for autologous stem cell transplant; (ix) anti-PD-1/PD-L1 experienced recurrent and/or metastatic HNSCC (irrespective of HPV status) with no curative options; (x) anti-PD-1/PD-L1 experienced locally advanced or metastatic CSCC not appropriate for surgery; and (xi) ≥1% LAG-3 expression in tumor tissue. The tumor tissue can comprise tumor cells and/or tumor-infiltrating immune cells.

In certain embodiments, methods are provided for treating cancer or inhibiting the growth of a tumor. The methods, according to this aspect, comprise: (1) selecting a patient with a tumor, wherein the selected patient has not received prior treatment with a PD-1 inhibitor or PD-L1 inhibitor; and (2) administering to the patient (a) 350 mg anti-PD-1 antibody comprising the HCVR/LCVR amino acid sequence pair of SEQ ID NOs: 1/2; and (b) 1, 3, 10, 20, or 40 mg/kg or 1600 mg anti-LAG-3 antibody comprising the HCVR/LCVR amino acid sequence pair of SEQ ID NOs: 11/12. In some aspects, the administering of step (2) occurs once every 3 weeks, or once every 6 weeks. In some aspects, the selecting of step (1) further identifies a patient as satisfying one or more of the following criteria: (i) ineligible for platinum based therapy, or who have had tumor progression or recurrence within 6 months of last dose of platinum therapy; (ii) confirmed diagnosis of malignancy; (iii) demonstrated progression of a tumor for which there is no available therapy likely to convey clinical benefit; (iv) anti-PD-1/PD-L1 naïve stage IIIB, IIIC, or IV NSCLC either without prior therapy for metastatic disease; (v) disease progression/recurrence after one platinum-containing regimen; (vi) anti-PD-1/PD-L1 naïve advanced or metastatic ccRCC with a clear cell component who had received no more than 2 previous regimens of anti-angiogenic therapy; (vii) anti-PD-1/PD-L1 naïve metastatic TNBC (estrogen, progesterone, and human epidermal growth factor receptor 2 negative) who have received 5 or fewer prior lines of therapy; (viii) anti-PD-1/PD-L1 naïve advanced or metastatic non-uveal melanoma who have received no more than 2 previous regimens for metastatic disease; (ix) anti-PD-1/PD-L1 naïve relapsed/refractory DLBCL who have either progressed after or are not candidates for autologous stem cell transplant; (x) anti-PD-1/PD-L1 naïve recurrent and/or metastatic HNSCC (irrespective of HPV status) with no curative options; (xi) anti-PD-1/PD-L1 naïve locally advanced or metastatic CSCC not appropriate for surgery; and (xii) the patient has ≥1% LAG-3 expression in tumor tissue. The tumor tissue can comprise tumor cells and/or tumor-infiltrating immune cells.

In certain embodiments, methods are provided for treating cancer or inhibiting the growth of a tumor. The methods, according to this aspect, comprise: (1) selecting a patient with a tumor; and (2) administering to the patient (a) 1, 3, 10, 20, or 40 mg/kg or 1600 mg anti-LAG-3 antibody comprising the HCVR/LCVR amino acid sequence pair of SEQ ID NOs: 11/12 as a monotherapy for about one month to about twelve months; then further administering to the patient, in combination with (a), (b) 350 mg anti-PD-1 antibody comprising the HCVR/LCVR amino acid sequence pair of SEQ ID NOs: 1/2. In some aspects, the administering of step (2) occurs once every 3 weeks, or once every 6 weeks.

In certain embodiments, methods are provided for treating cancer or inhibiting the growth of a tumor. The methods, according to this aspect, comprise: administering to a patient in need thereof: (1) an initial loading dose comprising an anti-PD-1 antibody comprising the HCVR/LCVR amino acid sequence pair of SEQ ID NOs: 1/2; and an anti-LAG-3 antibody comprising the HCVR/LCVR amino acid sequence pair of SEQ ID NOs: 11/12; and (2) one or more secondary doses, wherein the one or more secondary doses occur one to four weeks after the immediately preceding dose. In some aspects, the method further comprises administering to a patient in need thereof: (3) one or more tertiary doses, wherein the one or more tertiary doses occur three to twelve weeks after the immediately preceding dose. In some aspects, the one or more secondary doses occur three weeks after the immediately preceding dose. In some aspects, the one or more tertiary doses occur three weeks or six weeks after the immediately preceding dose. In some aspects, the initial loading dose comprises (a) 500 mg to 1500 mg anti-PD-1 antibody and (b) 20 or 40 mg/kg anti-LAG-3 antibody. In some aspects, the one or more secondary doses comprise: (a) 350 mg anti-PD-1 antibody and (b) 1, 3, 10, 20, or 40 mg/kg anti-LAG-3 antibody. In some aspects, the one or more tertiary doses comprise: (a) 350 mg anti-PD-1 antibody and (b) 1, 3, 10, 20, or 40 mg/kg anti-LAG-3 antibody. In some aspects, the initial loading dose comprises (a) 500 mg to 1500 mg anti-PD-1 antibody and (b) 50 mg to 8000 mg anti-LAG-3 antibody. In some aspects, the one or more secondary doses comprise: (a) 350 mg anti-PD-1 antibody and (b) 600 mg, 800 mg, 1000 mg, 1200 mg, 1400 mg, 1600 mg, or 2000 mg anti-LAG-3 antibody. In some aspects, the one or more tertiary doses comprise: (a) 350 mg anti-PD-1 antibody and (b) 600 mg, 800 mg, 1000 mg, 1200 mg, 1400 mg, 1600 mg, or 2000 mg anti-LAG-3 antibody.

In certain embodiments, the cancer or tumor is a selected from the group consisting of renal cancer, lung cancer, breast cancer, endometrial cancer, squamous cell carcinoma, melanoma, and lymphoma.

In certain embodiments, the cancer or tumor is a selected from the group consisting of astrocytoma, bladder cancer, blood cancer, blood cancer, bone cancer, brain cancer, breast cancer, cervical cancer, clear cell renal cell carcinoma, colorectal cancer, microsatellite-intermediate colorectal cancer, cutaneous squamous cell carcinoma, diffuse large B-cell lymphoma, endometrial cancer, esophageal cancer, fibrosarcoma, gastric cancer, glioblastoma, glioblastoma multiforme, head and neck squamous cell carcinoma, hepatic cell carcinoma, leukemia, liver cancer, leiomyosarcoma, lung cancer, lymphoma, melanoma, mesothelioma, myeloma, nasopharyngeal cancer, non-small cell lung cancer, osteosarcoma, ovarian cancer, pancreatic cancer, primary and/or recurrent cancer, prostate cancer, renal cell carcinoma, rhabdomyosarcoma, small cell lung cancer, squamous cell cancer, synovial sarcoma, thyroid cancer, triple negative breast cancer, uterine cancer, and Wilms' tumor. In some aspects, the cancer is a primary cancer. In some aspects, the cancer is metastatic and/or recurrent cancer.

In certain embodiments, each dose of anti-PD-1 antibody comprises 0.1-20 mg/kg of the subject's body weight. In certain embodiments, each dose of anti-PD-1 antibody comprises 0.3, 1, 3, or 10 mg/kg of the subject's body weight. In certain embodiments, each dose of the anti-PD-1 antibody comprises 50-1500 mg, for example, 350 mg. In some aspects, each dose of the anti-PD-1 antibody comprises 350 mg. In some aspects, the therapeutically effective amount of the anti-PD-1 antibody comprises between 50 to 1500 mg.

In certain embodiments, each dose of the anti-LAG-3 antibody comprises between 0.1 mg/kg and 50 mg/kg of the subject's body weight, for example, 1, 3, 10, 20, 30, or 40 mg/kg of the subject's body weight. In certain embodiments, each dose of the anti-LAG-3 antibody comprises between 50 and 8000 mg, for example, 1600 mg. In some aspects, the therapeutically effective amount of the anti-LAG-3 antibody comprises between 50 to 8000 mg.

In certain embodiments, each dose of the anti-PD-1 antibody comprises 0.3, 1, 3, or 10 mg/kg of the subject's body weight and each dose of the anti-LAG-3 antibody comprises 50 mg to 8000 mg. In certain embodiments, each dose of the anti-PD-1 antibody comprises 1, 3, or 10 mg/kg of the subject's body weight and each dose of the LAG-3 antibody comprises 100, 300, 1000, 1600, or 3000 mg. In certain embodiments, each dose of the anti-PD-1 antibody comprises 50 to 1500 mg, e.g. 100 mg, 150 mg, 200 mg, 250 mg, 300 mg, 350 mg, 400 mg, 450 mg, or 500 mg, and each dose of the anti-LAG-3 antibody comprises 100, 300, 400, 500, 600, 700, 800, 900, 1000, 1100, 1200, 1300, 1400, 1500, 1600, 1700, 1800, 1900, 2000, 2500, or 3000 mg. In certain embodiments, each dose of the anti-PD-1 antibody comprises 1, 3, or 10 mg/kg and each dose of the anti-LAG-3 antibody comprises 1, 3, 10, 20, 30, or 40 mg/kg of the subject's body weight. In certain embodiments, each dose of the anti-PD-1 antibody comprises 200 mg, 250 mg or 350 mg and each dose of the anti-LAG-3 antibody comprises 800 mg, 1000 mg, 1400 mg or 1600 mg.

In certain embodiments, the antibodies are administered intravenously, subcutaneously, or intraperitoneally.

In certain embodiments, the methods comprise administering a therapeutically effective amount of an anti-PD-1 antibody, or anti-PD-L1 antibody, prior to, concurrent with, or subsequent to an anti-LAG-3 antibody. In certain embodiments, the methods comprise administering a therapeutically effective amount of an anti-LAG-3 antibody prior to, concurrent with, or subsequent to an anti-PD-1, or anti-PD-L1 antibody. In one embodiment, the methods comprise administering an anti-LAG-3 antibody prior to an anti-PD-1 antibody. In some aspects, the anti-LAG-3 antibody can be administered as a first therapy, e.g. one to four dosages over several weeks or months, and then the anti-PD-1 antibody is administered as a co-therapy. In one embodiment, the methods comprise administering an anti-PD-1 antibody prior to an anti-LAG-3 antibody. In one embodiment, the methods comprise administering an anti-PD-1 antibody the same day as an anti-LAG-3 antibody. In some aspects, the anti-LAG-3 antibody and anti-PD-1 antibody are administered the same day, but sequentially, and the anti-LAG3 antibody is administered first.

In certain embodiments, the methods comprise administering multiple therapeutic doses each of an anti-PD-1 antibody and an anti-LAG-3 antibody over many months to years. The treatment interval (whether for monotherapy or combination therapy) can be from about 0.5 weeks to about 12 weeks, i.e. the treatments are administered about 0.5 weeks to about 12 weeks apart, for example, about 18 days. In other words, the treatments are administered 0.5 weeks to 12 weeks after the immediately preceding dose. For example, in some aspects, each dose of the anti-PD-1 antibody is administered 0.5 weeks to 12 weeks after the immediately preceding dose. In some aspects, each dose of the anti-LAG-3 antibody is administered 0.5 weeks to 12 weeks after the immediately preceding dose. In certain embodiments, each dose of the anti-PD-1 antibody is administered once a week, once in 2 weeks, once in 3 weeks, once in 4 weeks, or once in 6 weeks and each dose of the anti-LAG-3 antibody is administered once a week, once in 2 weeks, once in 3 weeks, once in 4 weeks, or once in 6 weeks. In one embodiment, each dose of the anti-PD-1 antibody is administered once in 3 weeks and each dose of the anti-LAG-3 antibody is administered once in 3 weeks. In certain embodiments, a patient can receive treatment once every 3 weeks for several months to several years, then can receive treatment every 6 weeks for several months to several years. In some embodiments, one or both of the anti-PD-1 antibody and the anti-LAG-3 antibody are administered until disease progression. In some embodiments, one or both of the anti-PD-1 antibody and the anti-LAG-3 antibody are administered until toxicity due to the treatment is unacceptable.

In certain embodiments, the anti-PD-1 antibody and the anti-LAG-3 antibody are administered in combination with a third therapeutic agent or therapy.

In some aspects, the further therapeutic agent or therapy is selected from the group consisting of radiation, surgery, a chemotherapeutic agent, a cancer vaccine, a PD-L1 inhibitor, a CTLA-4 inhibitor, a TIM3 inhibitor, a BTLA inhibitor, a TIGIT inhibitor, a CD47 inhibitor, a CD28 agonist, a CD38 inhibitor, an indoleamine-2,3-dioxygenase (IDO) inhibitor, a vascular endothelial growth factor (VEGF) antagonist, an angiopoietin-2 (Ang2) inhibitor, a transforming growth factor beta (TGFβ) inhibitor, an epidermal growth factor receptor (EGFR) inhibitor, an antibody to a tumor-specific antigen, a CD28 agonist, a GITR agonist, a 4-1 BB agonist, CD20×CD3 bispecific antibody (e.g., REGN1979), MUC16×CD3 bispecific antibody, Bacillus Calmette-Guerin vaccine, granulocyte-macrophage colony-stimulating factor, an oncolytic virus, a cytotoxin, an interleukin 6 receptor (IL-6R) inhibitor, an interleukin 4 receptor (IL-4R) inhibitor, an IL-10 inhibitor, IL-2, IL-7, IL-21, II-12, IL-15, an antibody-drug conjugate (ADC) (e.g., anti-CD19-DM4 ADC, and anti-DS6-DM4 ADC), chimeric antigen receptor T cells (e.g., CD19-targeted T cells) or other cell therapies, and an anti-inflammatory drug.

According to certain embodiments, the anti-PD-1 antibody or antigen-binding protein comprises the heavy chain complementarity determining regions (HCDRs) of a heavy chain variable region (HCVR) comprising the amino acid sequence of SEQ ID NO: 1 and the light chain CDRs of a light chain variable region (LCVR) comprising the amino acid sequence of SEQ ID NO: 2. One such type of antigen-binding protein that can be used in the context of the methods provided herein is an anti-PD-1 antibody such as REGN2810 (also known as cemiplimab, LIBTAYO®).

According to certain embodiments, the anti-PD-1 antibody or antigen-binding fragment thereof comprises the heavy chain complementarity determining regions (HCDR1, HCDR2 and HCDR3) of a heavy chain variable region (HCVR) and three light chain complementarity determining regions (LCDR1, LCDR2 and LCDR3) of a light chain variable region (LCVR), wherein HCDR1 comprises the amino acid sequence of SEQ ID NO: 3; HCDR2 comprises the amino acid sequence of SEQ ID NO: 4; HCDR3 comprises the amino acid sequence of SEQ ID NO: 5; LCDR1 comprises the amino acid sequence of SEQ ID NO: 6; LCDR2 comprises the amino acid sequence of SEQ ID NO: 7; and LCDR3 comprises the amino acid sequence of SEQ ID NO: 8.

In some aspects, the anti-PD-1 antibody or antigen-binding fragment thereof comprises an HCVR having the amino acid sequence of SEQ ID NO: 1 and an LCVR having the amino acid sequence of SEQ ID NO: 2.

In some aspects, the anti-PD-1 antibody comprises a heavy chain comprising the amino acid sequence of SEQ ID NO: 9 and a light chain comprising the amino acid sequence of SEQ ID NO: 10.

According to certain embodiments, the anti-LAG-3 antibody or antigen-binding protein comprises the heavy chain complementarity determining regions (HCDRs) of a heavy chain variable region (HCVR) comprising the amino acid sequence of SEQ ID NO: 11 and the light chain CDRs (LCDR1, LCDR2 and LCDR3) of a light chain variable region (LCVR) of SEQ ID NO: 12. On such type of antigen-binding protein that can be used in the context of the methods provided herein is an anti-LAG-3 antibody such as REGN3767.

According to certain embodiments, the anti-LAG-3 antibody or antigen-binding fragment thereof comprises the three heavy chain CDRs (HCDR1, HCDR2 and HCDR3) of a HCVR and three light chain CDRs (LCDR1, LCDR2 and LCDR3) of a LCVR, wherein HCDR1 comprises the amino acid sequence of SEQ ID NO: 13; HCDR2 comprises the amino acid sequence of SEQ ID NO: 14; HCDR3 comprises the amino acid sequence of SEQ ID NO: 15; LCDR1 comprises the amino acid sequence of SEQ ID NO: 16; LCDR2 comprises the amino acid sequence of SEQ ID NO: 17; and LCDR3 comprises the amino acid sequence of SEQ ID NO: 18.

In some aspects, the anti-LAG-3 antibody or antigen-binding fragment thereof comprises an HCVR having the amino acid sequence of SEQ ID NO: 11 and the LCVR having the amino acid sequence of SEQ ID NO: 12.

In some aspects, the anti-LAG-3 antibody comprises a heavy chain comprising the amino acid sequence of SEQ ID NO: 19 and a light chain comprising the amino acid sequence of SEQ ID NO: 20.

Further provided is a combination comprising: a PD-1 inhibitor in association with a LAG-3 inhibitor and optionally, a pharmaceutically acceptable carrier. For example, such combinations may include a co-formulation or the inhibitors can be in separate compositions. The PD-1 inhibitor and/or LAG-3 inhibitor is, in some embodiments, an antibody or antigen-binding fragment thereof that binds specifically to PD-1 and/or LAG-3, respectively. In some embodiments, the PD-1 inhibitor is an antibody or an antigen-binding fragment thereof that specifically binds to PD-1 and comprises REGN2810. In some embodiments, the LAG-3 inhibitor is an antibody or an antigen-binding fragment thereof that specifically binds to LAG-3 and comprises REGN3767.

In certain embodiments, the anti-PD-1 antibody or antigen-binding fragment thereof and the anti-LAG-3 antibody or antigen-binding fragment thereof can be formulated separately or in combination. As such, the anti-PD1 antibody or antigen-binding fragment thereof and the anti-LAG-3 antibody or antigen binding-fragment thereof can be combined and used in the manufacture of a medicament to treat or inhibit the growth of cancer in a subject, including humans.

In certain embodiments, the cancer is astrocytoma, bladder cancer, blood cancer, blood cancer, bone cancer, brain cancer, breast cancer, cervical cancer, clear cell renal cell carcinoma, colorectal cancer, microsatellite-intermediate colorectal cancer, cutaneous squamous cell carcinoma, diffuse large B-cell lymphoma, endometrial cancer, esophageal cancer, fibrosarcoma, gastric cancer, glioblastoma, glioblastoma multiforme, head and neck squamous cell carcinoma, hepatic cell carcinoma, leukemia, liver cancer, leiomyosarcoma, lung cancer, lymphoma, melanoma, mesothelioma, myeloma, nasopharyngeal cancer, non-small cell lung cancer, osteosarcoma, ovarian cancer, pancreatic cancer, primary and/or recurrent cancer, prostate cancer, renal cell carcinoma, rhabdomyosarcoma, small cell lung cancer, squamous cell cancer, synovial sarcoma, thyroid cancer, triple negative breast cancer, uterine cancer, or Wilms' tumor. In some aspects, the cancer is a primary cancer. In some aspects, the cancer is metastatic and/or recurrent cancer.

In certain embodiments, the subject or patient has received prior anti-cancer therapy comprising one or more of a PD-1 inhibitor, a PD-L1 inhibitor, surgery, radiation therapy or chemotherapy. In some aspects, the prior anti-cancer therapy comprises a PD-1 inhibitor or a PD-L1 inhibitor. In certain embodiments, the subject is resistant or inadequately responsive to, or relapsed after prior therapy.

In certain embodiments, the subject has not received prior anti-cancer therapy.

Such combinations optionally include one or more further therapeutic agents or therapy.

Also provided herein is an injection device (e.g., hypodermic needle and syringe, an autoinjector or a pre-filled syringe) or vessel (e.g., a vial) that includes a combination of the antibodies (e.g., REGN2810/REGN3767).

Further provided are methods for administering the combination of the present disclosure to a subject (e.g., a human suffering from cancer) including the step of introducing the components of the combination into the body of the subject, e.g., parenterally, for example, by injection using an injection device according to the present disclosure.

Other embodiments will become apparent from a review of the ensuing detailed description.

### BRIEF DESCRIPTION OF THE FIGURES

FIG. 1 depicts a study flow diagram for an individual patient.

FIG. 2 depicts a study design diagram showing the dose escalation scheme and cohorts.

FIG. 3A and FIG. 3B provide an analysis of T cell subset proliferation following initiation of REGN3767 monotherapy or REGN3767+Cemiplimab.

### DETAILED DESCRIPTION

It is to be understood that this invention is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H1}, C_{H2} and C_{H3}. Each light chain comprises a light chain variable region (abbreviated herein as LCVR or V_{L}) and a light chain constant region. The light chain constant region comprises one domain (C_{L1}). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-IL-4R antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H1}; (ii) V_{H}-C_{H2}; (iii) V_{H}-C_{H3}, (iv) V_{H}-C_{H1}-C_{H2}, (V) V_{H}-C_{H1}-C_{H2}—C_{H3}, (VI) V_{H}-C_{H2}-C_{H3}; (Vii) V_{H}-C_{L}; (vii) V_{L}-C_{H1}, (ix) V_{L}-C_{H2}, (x) V_{L}-C_{H3}; (xi) V_{L}-C_{H1}-C_{H2}; (XII) V_{L}-C_{H1}-C_{H2}—C_{H3}; (Xiii) V_{L}-C_{H2}-C_{H3}; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen-binding fragment of an antibody of the present disclosure using routine techniques available in the art. For example, the present disclosure includes methods comprising the use of bispecific antibodies wherein one arm of an immunoglobulin is specific for PD-1 or LAG-3, or fragments thereof, and the other arm of the immunoglobulin is specific for a second therapeutic target or is conjugated to a therapeutic moiety. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

The antibodies used in the methods of the present disclosure may be human antibodies. The term “human antibody,” as used herein, is intended to include antibodies having variable and constant regions derived from human germline immunoglobulin sequences. The human antibodies of the disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods of the present disclosure may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Although any methods and materials similar or equivalent to those described herein can be used in the practice of the present disclosure, exemplary methods and materials are now described. All publications mentioned herein are incorporated herein by reference in their entirety.

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3rd ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, N.Y.; Kay et al. (1996) Phage Display of Peptides and Proteins: A Laboratory Manual, Academic Press, San Diego, Calif.; de Bruin et al. (1999) Nature Biotechnol. 17:397-399). Single chain antibodies and diabodies are described (see, e.g., Malecki et al. (2002) Proc. Natl. Acad. Sci. USA 99:213-218; Conrath et al. (2001) J. Biol. Chem. 276:7346-7350; Desmyter et al. (2001) J. Biol. Chem. 276:26285-26290; Hudson and Kortt (1999) J. Immunol. Methods 231:177-189; and U.S. Pat. No. 4,946,778). Bifunctional antibodies are provided (see, e.g., Mack, et al. (1995) Proc. Natl. Acad. Sci. USA 92:7021-7025; Carter (2001) J. Immunol. Methods 248:7-15; Volkel, et al. (2001) Protein Engineering 14:815-823; Segal, et al. (2001) J. Immunol. Methods 248:1-6; Brennan, et al. (1985) Science 229:81-83; Raso, et al. (1997) J. Biol. Chem. 272:27623; Morrison (1985) Science 229:1202-1207; Traunecker, et al. (1991) EMBO J. 10:3655-3659; and U.S. Pat. Nos. 5,932,448, 5,532,210, and 6,129,914). Fully human antibodies may also be developed in genetically engineered mice such as the VelociMouse. See e.g., DeChiara et al., Producing fully ES cell-derived mice from eight-cell stage embryo injections, Methods Enzymol, 476:285-94 (2010); Dechiara et al., VelociMouse: fully ES cell-derived FO-generation mice obtained from the injection of ES cells into eight-cell-stage embryos. Methods Mol Biol, 530:311-24 (2009); U.S. Pat. Nos. 7,576,259; 7,659,442; or 7294754, and US2008/0078000A1.

Purification of antigen is not typically necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, N.J.; Givan (2001) Flow Cytometry, 2nd ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probes (2003) Catalogue, Molecular Probes, Inc., Eugene, Oreg.; Sigma-Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### PD-1 Inhibitors

According to certain exemplary embodiments of the present disclosure, the methods comprise administering a therapeutically effective amount of an anti-PD-1 antibody or antigen-binding fragment thereof. The term “PD-1” refers to the programmed death-1 protein, a T-cell co-inhibitor, also known as CD279. The amino acid sequence of full-length PD-1 is provided in GenBank as accession number NP_005009.2. PD-1 is a member of the CD28/CTLA-4/ICOS family of T-cell co-inhibitors. PD-1 is a 288-amino acid protein with an extracellular N-terminal domain which is IgV-like, a transmembrane domain and an intracellular domain containing an immunoreceptor tyrosine-based inhibitory (ITIM) motif and an immunoreceptor tyrosine-based switch (ITSM) motif (Chattopadhyay et al 2009, Immunol. Rev.). The PD-1 receptor has two ligands, PD-ligand-1 (PD-L1) and PD-L2.

PD-L1 is a 290 amino acid protein with an extracellular IgV-like domain, a transmembrane domain and a highly conserved intracellular domain of approximately 30 amino acids. PD-L1 is constitutively expressed on many cells such as antigen presenting cells (e.g., dendritic cells, macrophages, and B-cells) and on hematopoietic and non-hematopoietic cells (e.g., vascular endothelial cells, pancreatic islets, and sites of immune privilege). PD-L1 is also expressed on a wide variety of tumors, virally-infected cells and autoimmune tissue, and is a component of the immunosuppressive milieu (Ribas 2012, NEJM 366: 2517-2519).

PD-1 inhibitors include antibodies and antigen-binding fragments thereof and other substances (e.g., peptides and small molecules) that specifically bind to PD-1 and antagonize one or more biological activities of PD-1. Molecules that specifically bind to PD-1 may be referred to as “anti-PD-1”. In an embodiment of the disclosure, the PD-1 inhibitor is an antibody or antigen-binding fragment thereof that binds PD-L1 or PD-L2.

In an embodiment of the disclosure, the PD-1 inhibitor is an antibody or antigen-binding fragment thereof as set forth in U.S. Pat. No. 9,987,500.

According to certain embodiments, the antibodies used in the methods of the present disclosure specifically bind PD-1. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” PD-1, as used in the context of the present disclosure, includes antibodies that bind PD-1 or portion thereof with a K_{D} of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human PD-1 may, however, have cross-reactivity to other antigens, such as PD-1 molecules from other (non-human) species.

According to certain exemplary embodiments of the present disclosure, the anti-PD-1 antibody, or antigen-binding fragment thereof comprises a heavy chain variable region (HCVR), light chain variable region (LCVR), and/or complementarity determining regions (CDRs) comprising any of the amino acid sequences of the anti-PD-1 antibodies as set forth in U.S. Pat. No. 9,987,500.

In certain exemplary embodiments, the anti-PD-1 antibody or antigen-binding fragment thereof that can be used in the context of the methods of the present disclosure comprises the heavy chain complementarity determining regions (HCDRs) of a heavy chain variable region (HCVR) comprising the amino acid sequence of SEQ ID NO: 1 and the light chain complementarity determining regions (LCDRs) of a light chain variable region (LCVR) comprising the amino acid sequence of SEQ ID NO: 2. According to certain embodiments, the anti-PD-1 antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO: 3; the HCDR2 comprises the amino acid sequence of SEQ ID NO: 4; the HCDR3 comprises the amino acid sequence of SEQ ID NO: 5; the LCDR1 comprises the amino acid sequence of SEQ ID NO: 6; the LCDR2 comprises the amino acid sequence of SEQ ID NO: 7; and the LCDR3 comprises the amino acid sequence of SEQ ID NO: 8. In yet other embodiments, the anti-PD-1 antibody or antigen-binding fragment thereof comprises an HCVR comprising SEQ ID NO: 1 and an LCVR comprising SEQ ID NO: 2. In certain embodiments, the methods of the present disclosure comprise the use of an anti-PD-1 antibody, wherein the antibody comprises a heavy chain comprising the amino acid sequence of SEQ ID NO: 9. In some embodiments, the anti-PD-1 antibody comprises a light chain comprising the amino acid sequence of SEQ ID NO: 10. An exemplary antibody comprising a heavy chain variable region comprising the amino acid sequence of SEQ ID NO: 1 and a light chain variable region comprising the amino acid sequence of SEQ ID NO: 2 is the fully human anti-PD-1 antibody known as REGN2810 (cemiplimab; LIBTAYO®).

According to certain exemplary embodiments, the methods of the present disclosure comprise the use of REGN2810, or a bioequivalent thereof. The term “bioequivalent”, as used herein, refers to anti-PD-1 antibodies or PD-1-binding proteins or fragments thereof that are pharmaceutical equivalents or pharmaceutical alternatives whose rate and/or extent of absorption do not show a significant difference with that of REGN2810 when administered at the same molar dose under similar experimental conditions, either single dose or multiple dose. In the context of the disclosure, the term refers to antigen-binding proteins that bind to PD-1 which do not have clinically meaningful differences with REGN2810 in their safety, purity and/or potency.

Other anti-PD-1 antibodies that can be used in the context of the methods of the present disclosure include, e.g., the antibodies referred to and known in the art as nivolumab (U.S. Pat. No. 8,008,449), pembrolizumab (U.S. Pat. No. 8,354,509), MEDI0608 (U.S. Pat. No. 8,609,089), pidilizumab (U.S. Pat. No. 8,686,119), or any of the anti-PD-1 antibodies as set forth in U.S. Pat. Nos. 6,808,710, 7,488,802, 8,168,757, 8,354,509, 8,779,105, or 8900587. In an embodiment of the disclosure, a PD-1 inhibitor is as set forth in any of U.S. 20110008369, U.S. 20130017199, U.S. 20130022595, WO2006121168, WO20091154335, WO2012145493, WO2013014668, WO2009101611, EP2262837, and EP2504028.

The anti-PD-1 antibodies used in the context of the methods of the present disclosure may have pH-dependent binding characteristics. For example, an anti-PD-1 antibody for use in the methods of the present disclosure may exhibit reduced binding to PD-1 at acidic pH as compared to neutral pH. Alternatively, an anti-PD-1 antibody of the disclosure may exhibit enhanced binding to its antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding to PD-1 at acidic pH as compared to neutral pH” is expressed in terms of a ratio of the K_{D} value of the antibody binding to PD-1 at acidic pH to the K_{D} value of the antibody binding to PD-1 at neutral pH (or vice versa). For example, an antibody or antigen-binding fragment thereof may be regarded as exhibiting “reduced binding to PD-1 at acidic pH as compared to neutral pH” for purposes of the present disclosure if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present disclosure can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0, or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained. As used herein, the expression “acidic pH” means a pH of 6.0 or less.

### LAG-3 Inhibitors

The term “LAG-3” refers to the lymphocyte activation gene-3 protein, an immune checkpoint receptor or T cell co-inhibitor, also known as CD223. The amino acid sequence of full-length LAG-3 is provided in GenBank as accession number NP_002277.4. LAG-3 is a member of the immunoglobulin (Ig) superfamily. LAG-3 is a 503-amino acid type-1 transmembrane protein with four extracellular Ig-like domains D1 to D4 and is expressed on activated T cells, natural killer cells, B cells, plasmacytoid dendritic cells, and regulatory T cells. The LAG-3 receptor binds to MHC class II molecules present on antigen presenting cells (APCs).

As used herein, the term “T cell co-inhibitor” refers to a ligand and/or receptor which modulates the immune response via T cell activation or suppression. The term “T cell co-inhibitor”, also known as T cell co-signaling molecule, includes, but is not limited to, programmed death-1 (PD-1), cytotoxic T-lymphocyte antigen-4 (CTLA-4), B and T lymphocyte attenuator (BTLA), CD-28, 2B4, LY108, T cell immunoglobulin and mucin 3(TIM3), T cell immunoreceptor with immunoglobulin and ITIM (TIGIT; also known as VSIG9), leucocyte associated immunoglobulin-like receptor 1 (LAIR1; also known as CD305), inducible T cell costimulator (ICOS; also known as CD278), V-domain Ig suppressor of T cell activation (VISTA) and CD160.

LAG-3 inhibitors include antibodies and antigen-binding fragments thereof and other substances (e.g., peptides and small molecules) that specifically bind to LAG-3 and antagonize one or more biological activities of LAG-3. Molecules that specifically bind to LAG-3 may be referred to as “anti-LAG-3”.

In an embodiment of the disclosure, the LAG-3 inhibitor is an antibody or antigen-binding fragment thereof as set forth in U.S. 20170101472.

According to certain embodiments, the antibodies used in the methods of the present disclosure specifically bind LAG-3. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” LAG-3, as used in the context of the present disclosure, includes antibodies that bind LAG-3 or portion thereof with a K_{D} of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human LAG-3 may, however, have cross-reactivity to other antigens, such as LAG-3 molecules from other (non-human) species.

According to certain exemplary embodiments of the present disclosure, the anti-LAG-3 antibody, or antigen-binding fragment thereof comprises a heavy chain variable region (HCVR), light chain variable region (LCVR), and/or complementarity determining regions (CDRs) comprising any of the amino acid sequences of the anti-LAG-3 antibodies as set forth in U.S. 20170101472.

In certain exemplary embodiments, the anti-LAG-3 antibody or antigen-binding fragment thereof that can be used in the context of the methods of the present disclosure comprises the heavy chain complementarity determining regions (HCDRs) of a heavy chain variable region (HCVR) comprising the amino acid sequence of SEQ ID NO: 11 and the light chain complementarity determining regions (LCDRs) of a light chain variable region (LCVR) comprising the amino acid sequence of SEQ ID NO: 12. According to certain embodiments, the anti-LAG-3 antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO: 13; the HCDR2 comprises the amino acid sequence of SEQ ID NO: 14; the HCDR3 comprises the amino acid sequence of SEQ ID NO: 15; the LCDR1 comprises the amino acid sequence of SEQ ID NO: 16; the LCDR2 comprises the amino acid sequence of SEQ ID NO: 17; and the LCDR3 comprises the amino acid sequence of SEQ ID NO: 18. In yet other embodiments, the anti-LAG-3 antibody or antigen-binding fragment thereof comprises an HCVR comprising SEQ ID NO: 11 and an LCVR comprising SEQ ID NO: 12. In certain embodiments, the methods of the present disclosure comprise the use of an anti-LAG-3 antibody, wherein the antibody comprises a heavy chain comprising the amino acid sequence of SEQ ID NO: 19. In some embodiments, the anti-LAG-3 antibody comprises a light chain comprising the amino acid sequence of SEQ ID NO: 20. An exemplary antibody comprising a heavy chain variable region comprising the amino acid sequence of SEQ ID NO: 11 and a light chain variable region comprising the amino acid sequence of SEQ ID NO: 12 is the fully human anti-LAG-3 antibody known as REGN3767.

According to certain exemplary embodiments, the methods of the present disclosure comprise the use of REGN3767, or a bioequivalent thereof. The term “bioequivalent”, as used herein, refers to anti-LAG-3 antibodies or LAG-3-binding proteins or fragments thereof that are pharmaceutical equivalents or pharmaceutical alternatives whose rate and/or extent of absorption do not show a significant difference with that of REGN3767 when administered at the same molar dose under similar experimental conditions, either single dose or multiple dose. In the context of the disclosure, the term refers to antigen-binding proteins that bind to LAG-3 which do not have clinically meaningful differences with REGN3767 in their safety, purity and/or potency.

Other anti-LAG-3 antibodies that can be used in the context of the methods of the present disclosure include, e.g., the antibodies referred to and known in the art as relatlimab (U.S. 20110150892), LAG525 (WO2017/037203), GSK2831781 (U.S. 2016/0017037), Sym022 (WO2018/069500), INCAGN02385 (U.S. 20180127499) or any of the anti-LAG-3 antibodies as set forth in US Patent/Publication Nos. 5976877, 6143273, 6197524, 8551481, 20110070238, 20110150892, 20130095114, 20140093511, 20140127226, 20140286935, and in WO95/30750, WO97/03695, WO98/58059, WO2004/078928, WO2008/132601, WO2010/019570, WO2014/008218, EP0510079B1, EP0758383B1, EP0843557B1, EP0977856B1, EP1897548B2, EP2142210A1, and EP2320940B1.

### Methods for Treating Cancer or Inhibiting Tumor Growth

The present disclosure includes methods for treating, ameliorating or reducing the severity of at least one symptom or indication, or inhibiting the growth of a cancer in a subject. The methods according to this aspect comprise administering a therapeutically effective amount of an antibody or antigen-binding fragment thereof that specifically binds PD-1 in combination with a therapeutically effective amount of an antibody or antigen-binding fragment thereof that specifically binds LAG-3 to a subject in need thereof. As used herein, the terms “treat”, “treating”, or the like, mean to alleviate symptoms, eliminate the causation of symptoms either on a temporary or permanent basis, to delay or inhibit tumor growth, to reduce tumor cell load or tumor burden, to promote tumor regression, to cause tumor shrinkage, necrosis and/or disappearance, to prevent tumor recurrence, and/or to increase duration of survival of the subject.

As used herein, the expression “a subject in need thereof” means a human or non-human mammal that exhibits one or more symptoms or indications of cancer, and/or who has been diagnosed with cancer, and who needs treatment for the same. In many embodiments, the term “subject” may be interchangeably used with the term “patient”. For example, a human subject may be diagnosed with a primary or a metastatic tumor and/or with one or more symptoms or indications including, but not limited to, enlarged lymph node(s), swollen abdomen, chest pain/pressure, unexplained weight loss, fever, night sweats, persistent fatigue, loss of appetite, enlargement of spleen, itching. In specific embodiments, the expression includes human subjects that have and need treatment for astrocytoma, bladder cancer, blood cancer, blood cancer, bone cancer, brain cancer, breast cancer, cervical cancer, clear cell renal cell carcinoma, colorectal cancer, microsatellite-intermediate colorectal cancer, cutaneous squamous cell carcinoma, diffuse large B-cell lymphoma, endometrial cancer, esophageal cancer, fibrosarcoma, gastric cancer, glioblastoma, glioblastoma multiforme, head and neck squamous cell carcinoma, hepatic cell carcinoma, leukemia, liver cancer, leiomyosarcoma, lung cancer, lymphoma, melanoma, mesothelioma, myeloma, nasopharyngeal cancer, non-small cell lung cancer, osteosarcoma, ovarian cancer, pancreatic cancer, primary and/or recurrent cancer, prostate cancer, renal cell carcinoma, rhabdomyosarcoma, small cell lung cancer, squamous cell cancer, synovial sarcoma, thyroid cancer, triple negative breast cancer, uterine cancer, or Wilms' tumor. In certain embodiments, the expression “a subject in need thereof” includes patients with a cancer that is resistant to or refractory to or is inadequately controlled by prior therapy (e.g., treatment with a conventional anti-cancer agent or therapy such as radiation, chemotherapy or surgery, or treatment with an anti-cancer biologic). For example, the expression includes subjects who have been treated with a PD-1 or PD-L1 inhibitor (e.g., an anti-PD-1 antibody). The expression also includes subjects with a cancer for which conventional anti-cancer therapy is inadvisable, for example, due to toxic side effects. For example, the expression includes patients who have received one or more cycles of chemotherapy with toxic side effects. In certain embodiments, the expression “a subject in need thereof” includes patients with a cancer which has been treated but which has subsequently relapsed or metastasized. For example, patients with cancer that may have received treatment with one or more anti-cancer agents leading to tumor regression; however, subsequently have relapsed with cancer resistant to the one or more anti-cancer agents (e.g., chemotherapy-resistant cancer) are treated with the methods provided herein.

The expression “a subject in need thereof” also includes subjects who are at risk of developing a cancer, e.g., persons with a family history of cancer, persons with a past cancer occurrence, or persons with an immune system compromised. In some aspects, the subject is resistant or inadequately responsive to, or relapsed after prior therapy.

In certain embodiments, the methods provided herein may be used to treat patients that show elevated levels of one or more cancer-associated biomarkers (e.g., PD-L1, or LAG-3). For example, the methods of the present invention comprise administering a therapeutically effective amount of an anti-LAG-3 antibody in combination with an anti-PD-1 antibody to a patient with an elevated level of LAG-3 and/or PD-L1. In one embodiment, the present methods are used in patients with cancer that are selected on the basis of LAG-3 expression in cancer tissue, wherein the cancer tissue comprises tumor cells and tumor-infiltrating immune cells. In certain embodiments, the present methods are used to treat patients with a cancer wherein the patients are selected on the basis of 1% LAG-3 expression in cancer tissue and/or immune cells. In one embodiment, the present methods are used in patients with cancer that are selected on the basis of LAG-3 expression in cancer tissue, wherein the cancer tissue comprises tumor cells and tumor-infiltrating immune cells. In certain embodiments, the present methods are used to treat patients with a cancer wherein the patients are selected on the basis of 1% LAG-3 expression in cancer tissue and/or immune cells. Methods to determine LAG-3 or PD-L1 expression in cancer tissue and/or tumor-associated immune cells are well-known in the art. In certain embodiments, the expression of LAG-3 in tumor tissue is determined by any assay known in the art, for example, by an ELISA assay or by an immunohistochemistry (IHC) assay (e.g., as described in He et al 2017, J. Thoracic Oncol. 12: 814-823; WO2016124558 or WO2016191751). In certain embodiments, the expression of LAG-3 or PD-L1 is determined by quantitating RNA expression, for example, by in situ hybridization or by RT-PCR. In certain embodiments, the expression of LAG-3 is determined by imaging with a labeled anti-LAG-3 antibody, for example, by immuno-positron emission tomography or iPET [See, e.g., The Oncologist, 12: 1379 (2007); Journal of Nuclear Medicine, 52(8): 1171 (2011); US Patent Application Publication 2018/0228926]. In certain embodiments, the expression of PD-L1 is determined by imaging with a labeled anti-PD-L1 antibody, for example, by immuno-positron emission tomography or iPET (US Patent Application Publication 2018/0161464).

In certain embodiments, the methods provided herein are used in a subject with a cancer. The terms “tumor”, “cancer” and “malignancy” are interchangeably used herein. Examples of cancer include, but are not limited to, astrocytoma, bladder cancer, blood cancer, blood cancer, bone cancer, brain cancer, breast cancer, cervical cancer, clear cell renal cell carcinoma, colorectal cancer, microsatellite-intermediate colorectal cancer, cutaneous squamous cell carcinoma, diffuse large B-cell lymphoma, endometrial cancer, esophageal cancer, fibrosarcoma, gastric cancer, glioblastoma, glioblastoma multiforme, head and neck squamous cell carcinoma, hepatic cell carcinoma, leukemia, liver cancer, leiomyosarcoma, lung cancer, lymphoma, melanoma, mesothelioma, myeloma, nasopharyngeal cancer, non-small cell lung cancer, osteosarcoma, ovarian cancer, pancreatic cancer, primary and/or recurrent cancer, prostate cancer, renal cell carcinoma, rhabdomyosarcoma, small cell lung cancer, squamous cell cancer, synovial sarcoma, thyroid cancer, triple negative breast cancer, uterine cancer, and Wilms' tumor. In some aspects, the cancer is a primary cancer. In some aspects, the cancer is metastatic and/or recurrent cancer.

In certain embodiments, the cancer or tumor is a selected from the group consisting of melanoma, clear cell renal cancer, non-small cell lung cancer, triple negative breast cancer, endometrial cancer, cutaneous squamous cell carcinoma, diffuse large B-cell lymphoma, and head and neck squamous cell carcinoma.

According to certain embodiments, the present disclosure includes methods for treating, or delaying or inhibiting the growth of a tumor. In certain embodiments, this includes methods to promote tumor regression. In certain embodiments, this includes methods to reduce tumor cell load or to reduce tumor burden. In certain embodiments, the present disclosure includes methods to prevent tumor recurrence. The methods, according to this aspect, comprise sequentially administering a therapeutically effective amount of an anti-PD-1 antibody in combination with anti-LAG-3 antibody to a subject in need thereof, wherein each antibody is administered to the subject in multiple doses, e.g., as part of a specific therapeutic dosing regimen. For example, the therapeutic dosing regimen may comprise administering one or more doses of an anti-PD-1 antibody to the subject at a frequency of about once a day, once every two days, once every three days, once every four days, once every five days, once every six days, once a week, once every two weeks, once every three weeks, once every four weeks, once a month, once every six weeks, once every two months, once every three months, once every four months, or less frequently. In certain embodiments, the one or more doses of anti-PD-1 antibody are administered in combination with one or more doses of a therapeutically effective amount of anti-LAG-3 antibody, wherein the one or more doses of the anti-LAG-3 antibody are administered to the subject at a frequency of about once a day, once every two days, once every three days, once every four days, once every five days, once every six days, once a week, once every two weeks, once every three weeks, once every four weeks, once a month, once every six weeks, once every two months, once every three months, once every four months, or less frequently.

In certain embodiments, the present disclosure includes methods to inhibit, retard or stop tumor metastasis or tumor infiltration into peripheral organs. The methods, according to this aspect, comprise administering a therapeutically effective amount of an anti-PD-1 antibody to a subject in need thereof. In certain embodiments, the anti-PD-1 antibody is administered in combination with an anti-LAG-3 antibody.

In specific embodiments, the present disclosure provides methods for increased anti-tumor efficacy or increased tumor inhibition. In certain embodiments, the methods provide for increased tumor inhibition, e.g., by about 20%, more than 20%, more than 30%, more than 40% more than 50%, more than 60%, more than 70% or more than 80% as compared to a subject administered either antibody as a monotherapy.

The methods provided herein, according to certain embodiments, comprise administering to a subject with cancer a therapeutically effective amount of an anti-PD-1 antibody prior to, concurrent with, or after administering a therapeutically effective amount of anti-LAG-3 antibody. In some aspects, the anti-PD-1 antibody may be administered about 1 day, more than 1 day, more than 2 days, more than 3 days, more than 4 days, more than 5 days, more than 6 days, more than 7 days, or more than 8 days prior to the anti-LAG-3 antibody. In some aspects, the anti-PD-1 antibody and anti-LAG-3 antibody are administered concurrently, or within 30 minutes, or within 60 minutes, or within 2 hours, or within 3 hours, or within a day of each other.

In certain embodiments, the methods provided herein comprise administering a therapeutically effective amount of an anti-PD-1 antibody to a subject with cancer. In specific embodiments, the cancer is indolent or aggressive. In certain embodiments, the subject is not responsive to prior therapy or has relapsed after prior therapy. Prior therapy can include surgery, radiation, and/or chemotherapy, or treatment with a PD-1 inhibitor, a PD-L1 inhibitor, and/or any other anti-cancer biologic.

In certain embodiments, the methods of the present disclosure comprise administering an anti-PD-1 antibody in combination with an anti-LAG-3 antibody to a subject in need thereof as a “first line” treatment (e.g., initial treatment). In other embodiments, an anti-PD-1 antibody in combination with anti-LAG-3 antibody is administered as a “second line” treatment (e.g., after prior therapy). For example, an anti-PD-1 antibody in combination with anti-LAG-3 antibody is administered as a “second line” treatment to a subject that has relapsed after prior therapy with, e.g., chemotherapy or rituximab.

In certain embodiments, the methods of the present disclosure are used to treat a patient with an MRD-positive disease. Minimum residual disease (MRD) refers to small numbers of cancer cells that remain in the patient during or after treatment, wherein the patient may or may not show symptoms or signs of the disease. Such residual cancer cells, if not eliminated, frequently lead to relapse of the disease. The present disclosure includes methods to inhibit and/or eliminate residual cancer cells in a patient upon MRD testing. MRD may be assayed according to methods known in the art (e.g., MRD flow cytometry). The methods, according to this aspect of the disclosure, comprise administering an anti-PD-1 antibody in combination with an anti-LAG-3 antibody to a subject in need thereof.

The methods provided herein, according to certain embodiments, comprise administering to a subject a therapeutically effective amount of each of an anti-PD-1 antibody and an anti-LAG-3 antibody in combination with a third therapeutic agent. The third therapeutic agent may be an agent selected from the group consisting of, e.g., radiation, chemotherapy, surgery, a cancer vaccine, CART, a PD-L1 inhibitor (e.g., an anti-PD-L1 antibody), an CD3 inhibitor, a CD20 inhibitor, a CTLA-4 inhibitor, a CD38 inhibitor, a TIM3 inhibitor, a BTLA inhibitor, a TIGIT inhibitor, a CD47 inhibitor, an indoleamine-2,3-dioxygenase (IDO) inhibitor, a vascular endothelial growth factor (VEGF) antagonist, an Ang2 inhibitor, a transforming growth factor beta (TGFβ) inhibitor, an epidermal growth factor receptor (EGFR) inhibitor, an antibody to a tumor-specific antigen [e.g., CA9, CA125, melanoma-associated antigen 3 (MAGE3), carcinoembryonic antigen (CEA)], Bacillus Calmette-Guerin vaccine, granulocyte-macrophage colony-stimulating factor, an oncolytic virus, a cytotoxin, a CD28 agonist, a GITR agonist, a 4-1BB agonist, CD20×CD3 bispecific antibody (e.g., REGN1979), MUC16×CD3 bispecific antibody, vimentin, tumor-M2-PK, prostate-specific antigen (PSA), mucin-1, MART-1, and CA19-9), a vaccine (e.g., Bacillus Calmette-Guerin), granulocyte-macrophage colony-stimulating factor, a cytotoxin, a chemotherapeutic agent, an IL-6R inhibitor, an IL-4R inhibitor, an IL-10 inhibitor, a cytokine such as IL-2, IL-7, IL-12, IL-21, and IL-15, an anti-inflammatory drug such as corticosteroids, and non-steroidal anti-inflammatory drugs.

In certain embodiments, the antibodies may be administered in combination with therapy including a chemotherapeutic agent, radiation or surgery. As used herein, the phrase “in combination with” means that the antibodies are administered to the subject at the same time as, just before, or just after administration of the third therapeutic agent. In certain embodiments, the third therapeutic agent is administered as a co-formulation with the antibodies. In a related embodiment, the present disclosure includes methods comprising administering a therapeutically effective amount of an anti-PD-1 antibody in combination with an anti-LAG-3 antibody to a subject who is on a background anti-cancer therapeutic regimen. The background anti-cancer therapeutic regimen may comprise a course of administration of, e.g., a chemotherapeutic agent, or radiation. The anti-PD-1 antibody in combination with the anti-LAG-3 antibody may be added on top of the background anti-cancer therapeutic regimen. In some embodiments, the antibodies are added as part of a “background step-down” scheme, wherein the background anti-cancer therapy is gradually withdrawn from the subject over time (e.g., in a stepwise fashion) while the antibodies are administered to the subject at a constant dose, or at an increasing dose, or at a decreasing dose, over time.

In certain embodiments, the methods of the present disclosure comprise administering to a subject in need thereof a therapeutically effective amount of an anti-PD-1 antibody in combination with a therapeutically effective amount of an anti-LAG-3 antibody, wherein administration of the antibodies leads to increased inhibition of tumor growth. In certain embodiments, tumor growth is inhibited by at least about 10%, about 20%, about 30%, about 40%, about 50%, about 60%, about 70% or about 80% as compared to an untreated subject or a subject administered with either antibody as monotherapy. In certain embodiments, the administration of an anti-PD-1 antibody and/or anti-LAG-3 antibody to a subject leads to increased tumor regression, tumor shrinkage and/or disappearance. In certain embodiments, the administration of an anti-PD-1 antibody and/or an anti-LAG-3 antibody leads to delay in tumor growth and development, e.g., tumor growth may be delayed by about 3 days, more than 3 days, about 7 days, more than 7 days, at least 10 days, more than 15 days, more than 1 month, more than 3 months, more than 6 months, more than 1 year, more than 2 years, or more than 3 years as compared to an untreated subject or a subject treated with either antibody as monotherapy. In certain embodiments, administration of an anti-PD-1 antibody in combination with an anti-LAG-3 antibody prevents tumor recurrence and/or increases duration of survival of the subject, e.g., increases duration of survival by more than 15 days, more than 1 month, more than 3 months, more than 6 months, more than 12 months, more than 18 months, more than 24 months, more than 36 months, or more than 48 months than an untreated subject or a subject which is administered either antibody as monotherapy. In certain embodiments, administration of the antibodies in combination increases progression-free survival or overall survival. In certain embodiments, administration of an anti-PD-1 antibody in combination with an anti-LAG-3 antibody increases response and duration of response in a subject, e.g., by more than 2%, more than 3%, more than 4%, more than 5%, more than 6%, more than 7%, more than 8%, more than 9%, more than 10%, more than 20%, more than 30%, more than 40% or more than 50% over an untreated subject or a subject which has received either antibody as monotherapy. In certain embodiments, administration of an anti-PD-1 antibody and/or an anti-LAG-3 antibody to a subject with cancer leads to complete disappearance of all evidence of tumor cells (“complete response”). In certain embodiments, administration of an anti-PD-1 antibody and/or an anti-LAG-3 antibody to a subject with a cancer leads to at least 30% or more decrease in tumor cells or tumor size (“partial response”). In certain embodiments, administration of an anti-PD-1 antibody and/or an anti-LAG-3 antibody to a subject with cancer leads to complete or partial disappearance of tumor cells/lesions including new measurable lesions. Tumor reduction can be measured by any of the methods known in the art, e.g., X-rays, positron emission tomography (PET), computed tomography (CT), magnetic resonance imaging (MRI), cytology, histology, or molecular genetic analyses. In some aspects, administration of an anti-PD-1 antibody in combination with an anti-LAG-3 antibody to a patient population results in more patients responding to treatment, results in patient responses to treatment that are longer even without more patients responding, and/or the patients that do respond to therapy have deeper responses.

In certain embodiments, the combination of administered antibodies is safe and well-tolerated by a patient wherein there is no increase or a tolerable increase in an adverse side effect as compared to a patient administered with either antibody as monotherapy.

### Combination Therapies

The methods of the present disclosure, according to certain embodiments, comprise administering to the subject an anti-LAG-3 antibody in combination with an anti-PD-1 antibody. In certain embodiments, the methods of the present disclosure comprise administering the antibodies for additive or synergistic activity to treat cancer. As used herein, the expression “in combination with” means that the anti-LAG-3 antibody is administered before, after, or concurrent with the anti-PD-1 antibody. The term “in combination with” also includes sequential or concomitant administration of anti-PD-1 antibody and an anti-LAG-3 antibody. For example, when administered “before” the anti-LAG-3 antibody, the anti-PD-1 antibody may be administered more than 150 hours, about 150 hours, about 100 hours, about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of the anti-LAG-3 antibody. When administered “after” the anti-LAG-3 antibody, the anti-PD-1 antibody may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours, about 72 hours, or more than 72 hours after the administration of the anti-LAG-3 antibody. Administration “concurrent” with the anti-LAG-3 antibody means that the anti-PD-1 antibody is administered to the subject in a separate dosage form within less than 5 minutes (before, after, or at the same time) of administration of the anti-LAG-3 antibody, e.g. within 5 minutes of completion of anti-LAG-3 antibody infusion, or administered to the subject as a single combined dosage formulation comprising both the anti-PD-1 antibody and the anti-LAG-3 antibody. In some aspects, the anti-PD-1 antibody is administered the same day as the anti-LAG-3 antibody. In some aspects, the anti-PD-1 antibody and the anti-LAG-3 antibody are administered in a separate dosage form but within 8 hours of each other, for example, within 6 hours, or within 5 hours, or within 4 hours, or within 3 hours, or within 2 hours, or within 60 minutes of each other.

In certain embodiments, the methods provided herein comprise administration of a third therapeutic agent wherein the third therapeutic agent is an anti-cancer drug. As used herein, “anti-cancer drug” means any agent useful to treat cancer including, but not limited to, cytotoxins and agents such as antimetabolites, alkylating agents, anthracyclines, antibiotics, antimitotic agents, procarbazine, hydroxyurea, asparaginase, corticosteroids, mytotane (O,P′-(DDD)), biologics (e.g., antibodies and interferons) and radioactive agents. As used herein, “a cytotoxin or cytotoxic agent”, also refers to a chemotherapeutic agent and means any agent that is detrimental to cells. Examples include Taxol® (paclitaxel), temozolamide, cytochalasin B, gramicidin D, ethidium bromide, emetine, cisplatin, mitomycin, etoposide, tenoposide, vincristine, vinbiastine, coichicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In certain embodiments, the methods provided herein comprise administration of a third therapeutic agent selected from the group consisting of radiation, surgery, a cancer vaccine, a PD-L1 inhibitor (e.g., an anti-PD-L1 antibody), a CD20 inhibitor, a CD3 inhibitor, a CTLA-4 inhibitor (e.g., ipilimumab), a CD38 inhibitor, a TIM3 inhibitor, a BTLA inhibitor, a TIGIT inhibitor, a CD47 inhibitor, an antagonist of another T-cell co-inhibitor or ligand (e.g., an antibody to CD-28, 2B4, LY108, LAIR1, ICOS, CD160 or VISTA), an indoleamine-2,3-dioxygenase (IDO) inhibitor, a vascular endothelial growth factor (VEGF) antagonist [e.g., a “VEGF-Trap” such as aflibercept or other VEGF-inhibiting fusion protein as set forth in U.S. Pat. No. 7,087,411, or an anti-VEGF antibody or antigen binding fragment thereof (e.g., bevacizumab, or ranibizumab) or a small molecule kinase inhibitor of VEGF receptor (e.g., sunitinib, sorafenib, or pazopanib)], an Ang2 inhibitor (e.g., nesvacumab), a transforming growth factor beta (TGFβ) inhibitor, an epidermal growth factor receptor (EGFR) inhibitor (e.g., erlotinib, cetuximab), an agonist to a co-stimulatory receptor (e.g., an agonist to glucocorticoid-induced TNFR-related protein), an antibody to a tumor-specific antigen [e.g., CA9, CA125, melanoma-associated antigen 3 (MAGE3), carcinoembryonic antigen (CEA)], a CD28 agonist, a GITR agonist, a 4-1BB agonist, CD20×CD3 bispecific antibody (e.g., REGN1979), MUC16×CD3 bispecific antibody, vimentin, tumor-M2-PK, prostate-specific antigen (PSA), mucin-1, MART-1, and CA19-9), a vaccine (e.g., Bacillus Calmette-Guerin, a cancer vaccine), an adjuvant to increase antigen presentation (e.g., granulocyte-macrophage colony-stimulating factor), an oncolytic virus, a cytotoxin, a chemotherapeutic agent (e.g., dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, and vincristine), radiotherapy, an IL-6R inhibitor (e.g., sarilumab), an IL-4R inhibitor (e.g., dupilumab), an IL-10 inhibitor, a cytokine such as IL-2, IL-7, IL-12, IL-21, and IL-15, an antibody-drug conjugate (ADC) (e.g., anti-CD19-DM4 ADC, and anti-DS6-DM4 ADC), chimeric antigen receptor T cells (e.g., CD19-targeted T cells) or other cell therapies, and an anti-inflammatory drug (e.g., corticosteroids, and non-steroidal anti-inflammatory drugs).

In certain embodiments, the methods provided herein comprise administering an anti-PD-1 antibody and an anti-LAG-3 antibody in combination with radiation therapy/chemotherapy to generate long-term durable anti-tumor responses and/or enhance survival of patients with cancer.

In some embodiments, the methods of the disclosure comprise administering radiation therapy prior to, concomitantly or after administering an anti-PD-1 antibody and an anti-LAG-3 antibody to a cancer patient. For example, radiation therapy may be administered in one or more doses to tumor lesions after administration of one or more doses of the antibodies. In some embodiments, radiation therapy may be administered locally to a tumor lesion to enhance the local immunogenicity of a patient's tumor (adjuvinating radiation) and/or to kill tumor cells (ablative radiation) before or after systemic administration of an anti-PD-1 antibody and/or an anti-LAG-3 antibody. In certain embodiments, the antibodies may be administered in combination with radiation therapy and a chemotherapeutic agent (e.g., temozolomide or cyclophosphamide) or a VEGF antagonist (e.g., aflibercept).

### Pharmaceutical Compositions and Administration

Provided herein are methods which comprise administering an anti-PD-1 antibody in combination with an anti-LAG-3 antibody to a subject wherein the antibodies are contained within separate or combined (single) pharmaceutical composition. The pharmaceutical compositions of the disclosure may be formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262: 4429-4432). Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition of the present disclosure can be delivered subcutaneously or intravenously with a standard needle and syringe. In one embodiment, the syringe is a pre-filled syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition can be delivered in a controlled release system. In one embodiment, a pump may be used. In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

### Administration Regimens

The present disclosure includes methods comprising administering to a subject an anti-PD-1 antibody at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In certain embodiments, the present disclosure includes methods comprising administering to a subject an anti-LAG-3 antibody at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In certain embodiments, the methods involve the administration of an anti-PD-1 antibody in combination with an anti-LAG-3 antibody at a dosing frequency of about four times a week, twice a week, once a week, once every two weeks, once every three weeks, once every four weeks, once every five weeks, once every six weeks, once every eight weeks, once every nine weeks, once every twelve weeks, or less frequently so long as a therapeutic response is achieved.

According to certain embodiments of the present disclosure, multiple doses of an anti-PD-1 antibody in combination with an anti-LAG-3 antibody may be administered to a subject over a defined time course. The methods according to this aspect of the disclosure comprise sequentially administering to a subject one or more doses of an anti-PD-1 antibody in combination with one or more doses of an anti-LAG-3 antibody. As used herein, “sequentially administering” means that each dose of the antibody is administered to the subject at a different point in time, e.g., on different days separated by a predetermined interval (e.g., hours, days, weeks or months). The present disclosure includes methods which comprise sequentially administering to the patient a single initial dose of an anti-PD-1 antibody, followed by one or more secondary doses of the anti-PD-1 antibody, and optionally followed by one or more tertiary doses of the anti-PD-1 antibody. In certain embodiments, the methods further comprise sequentially administering to the patient a single initial dose of an anti-LAG-3 antibody, followed by one or more secondary doses of the anti-LAG-3 antibody, and optionally followed by one or more tertiary doses of the anti-LAG-3 antibody.

According to certain embodiments of the present disclosure, multiple doses of an anti-PD-1 antibody and an anti-LAG-3 antibody may be administered to a subject over a defined time course. The methods according to this aspect of the disclosure comprise sequentially administering to a subject multiple doses of an anti-PD-1 antibody and an anti-LAG-3 antibody. As used herein, “sequentially administering” means that each dose of the anti-PD-1 antibody in combination with the anti-LAG-3 antibody is administered to the subject at a different point in time, e.g., on different days separated by a predetermined interval (e.g., hours, days, weeks or months).

According to certain embodiments of the present disclosure, multiple doses of an anti-LAG-3 antibody can be administered to a subject for several months or years, once every 3 or 6 weeks, then the subject is administered the anti-PD-1 antibody in combination with the anti-LAG-3 antibody, for several months or years. In some aspects, the anti-LAG-3 antibody dosage is different as a monotherapy versus the combination therapy. In some aspects, the anti-LAG-3 antibody dosage is the same whether administered as a monotherapy or in combination with the anti-PD-1 antibody.

The terms “initial dose,” “secondary doses,” and “tertiary doses,” refer to the temporal sequence of administration. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the antibody (anti-PD-1 antibody or anti-LAG-3 antibody). In certain embodiments, however, the amount contained in the initial, secondary and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”). For example, an anti-PD-1 antibody may be administered to a patient with cancer at a loading dose of about 1-20 mg/kg followed by one or more maintenance doses of about 3 mg/kg of the patient's body weight.

In one exemplary embodiment of the present disclosure, each secondary and/or tertiary dose is administered ½ to 14 (e.g., ½, 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose,” as used herein, means, in a sequence of multiple administrations, the dose of anti-PD-1 antibody (and/or anti-LAG-3 antibody) which is administered to a patient prior to the administration of the very next dose in the sequence with no intervening doses.

The methods according some aspects may comprise administering to a patient any number of secondary and/or tertiary doses of an anti-PD-1 antibody (and/or anti-LAG-3 antibody). For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In certain embodiments, one or more doses of an anti-PD-1 antibody and/or an anti-LAG-3 antibody are administered at the beginning of a treatment regimen as “induction doses” on a more frequent basis (twice a week, once a week or once in 2 weeks) followed by subsequent doses (“consolidation doses” or “maintenance doses”) that are administered on a less frequent basis (e.g., once in 4-12 weeks).

In some embodiments, concomitant administration of anti-PD-1 antibody and the anti-LAG-3 antibody which is administered at a separate dosage at a similar or different frequency relative to the anti-PD-1 antibody is contemplated herein. In some embodiments, the anti-LAG-3 antibody is administered before, after or concurrently with the anti-PD-1 antibody. In certain embodiments, the anti-LAG-3 antibody is administered as a single dosage formulation with the anti-PD-1 antibody.

The present disclosure includes methods comprising sequential administration of an anti-PD-1 antibody in combination with an anti-LAG-3 antibody, to a patient to treat a cancer. In some embodiments, the present methods comprise administering one or more doses of an anti-PD-1 antibody followed by one or more doses of an anti-LAG-3 antibody. In certain embodiments, the present methods comprise administering a single dose of an anti-PD-1 antibody followed by one or more doses of an anti-LAG-3 antibody. In some embodiments, one or more doses of about 0.1 mg/kg to about 20 mg/kg of an anti-PD-1 antibody may be administered followed by one or more doses of about 0.1 mg/kg to about 50 mg/kg of the anti-LAG-3 antibody to inhibit tumor growth and/or to prevent tumor recurrence in a subject with cancer. In some embodiments, the anti-PD-1 antibody is administered at one or more doses followed by one or more doses of the anti-LAG-3 antibody resulting in increased anti-tumor efficacy (e.g., greater inhibition of tumor growth, increased prevention of tumor recurrence as compared to an untreated subject or a subject administered with either antibody as monotherapy).

The present disclosure also includes methods comprising sequential administration of an anti-LAG-3 antibody in combination with an anti-PD-1 antibody, to a patient to treat a cancer. In some embodiments, the present methods comprise administering one or more doses of an anti-LAG-3 antibody followed by one or more doses of an anti-PD-1 antibody. In certain embodiments, the present methods comprise administering a single dose of an anti-LAG-3 antibody followed by one or more doses of an anti-PD-1 antibody. In some embodiments, one or more doses of about 0.1 mg/kg to about 50 mg/kg of an anti-LAG-3 antibody may be administered followed by one or more doses of about 0.1 mg/kg to about 20 mg/kg of the anti-PD-1 antibody to inhibit tumor growth and/or to prevent tumor recurrence in a subject with cancer. In some embodiments, one or more doses of about 50 mg to about 8000 mg of an anti-LAG-3 antibody may be administered followed by one or more doses of about 50 mg to about 1500 mg of the anti-PD-1 antibody to inhibit tumor growth and/or to prevent tumor recurrence in a subject with cancer. In some embodiments, the anti-LAG-3 antibody is administered at one or more doses followed by one or more doses of the anti-PD-1 antibody resulting in increased anti-tumor efficacy (e.g., greater inhibition of tumor growth, increased prevention of tumor recurrence as compared to an untreated subject or a subject administered with either antibody as monotherapy).

### Dosage

The amount of anti-PD-1 antibody and/or anti-LAG-3 antibody administered to a subject according to the methods of the present disclosure is, generally, a therapeutically effective amount. As used herein, the phrase “therapeutically effective amount” means an amount of antibody (anti-PD-1 antibody or anti-LAG-3 antibody) that results in, or has the therapeutic effect of, one or more of: (a) a reduction in the severity or duration of a symptom of cancer; (b) inhibition of tumor growth, or an increase in tumor necrosis, tumor shrinkage and/or tumor disappearance; (c) delay in tumor growth and development; (d) inhibit or retard or stop tumor metastasis; (e) prevention of recurrence of tumor growth; (f) increase in survival of a subject with a cancer; and/or (g) a reduction in the use or need for conventional anti-cancer therapy (e.g., reduced or eliminated use of chemotherapeutic or cytotoxic agents) as compared to an untreated subject or a subject administered with either antibody as monotherapy.

In the case of an anti-PD-1 antibody, a therapeutically effective amount can be from about 0.05 mg to about 1500 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 700 mg, about 800 mg, about 900 mg, about 1000 mg, about 1050 mg, about 1100 mg, about 1200 mg, about 1300 mg, about 1400 mg, or about 1500 mg, of the anti-PD-1 antibody. In certain embodiments, 350 mg of an anti-PD-1 antibody is administered. In certain embodiments, 1050 mg of an anti-PD-1 antibody is administered.

In the case of an anti-LAG-3 antibody, a therapeutically effective amount can be from about 10 mg to about 8000 mg, e.g., about 10 mg, about 20 mg, about 50 mg, about 70 mg, about 100 mg, about 120 mg, about 150 mg, about 200 mg, about 250 mg, about 300 mg, about 350 mg, about 400 mg, about 450 mg, about 500 mg, about 550 mg, about 600 mg, about 700 mg, about 800 mg, about 900 mg, about 1000 mg, about 1050 mg, about 1100 mg, about 1500 mg, about 1600 mg, about 1700 mg, about 2000 mg, about 2050 mg, about 2100 mg, about 2200 mg, about 2500 mg, about 2700 mg, about 2800 mg, about 2900 mg, about 3000 mg, about 3200 mg, about 4000 mg, about 5000 mg, about 6000 mg, about 7000 mg, or about 8000 mg of the anti-LAG-3 antibody.

The amount of either anti-PD-1 antibody or anti-LAG-3 antibody contained within the individual doses may be expressed in terms of milligrams of antibody per kilogram of subject body weight (i.e., mg/kg). In certain embodiments, either anti-PD-1 antibody or anti-LAG-3 antibody used in the methods of the present disclosure may be administered to a subject at a dose of about 1 to about 50 mg/kg of subject body weight. For example, anti-PD-1 antibody may be administered at dose of about 0.1 mg/kg to about 20 mg/kg of a patient's body weight. The anti-LAG-3 antibody may be administered at a dose of about 0.1 mg/kg to about 50 mg/kg of a patient's body weight.

### EXAMPLES

The following examples are put forth so as to provide those of ordinary skill in the art with a complete disclosure and description of how to make and use the methods and compositions of the disclosure, and are not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric. Compositions and methods set forth in the Examples form part of the present disclosure.

### Example 1: Clinical Trial of Anti-PD-1 Antibody and Anti-LAG-3 Antibody in Patients with Advanced Malignancies

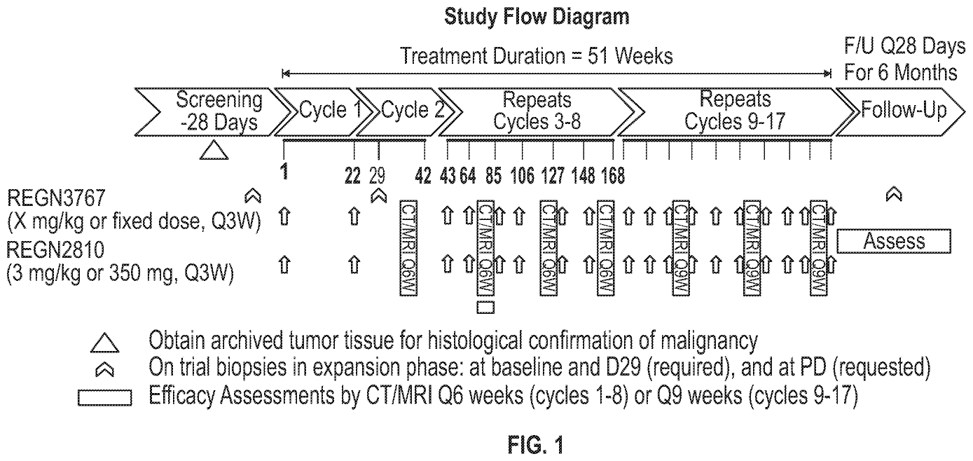

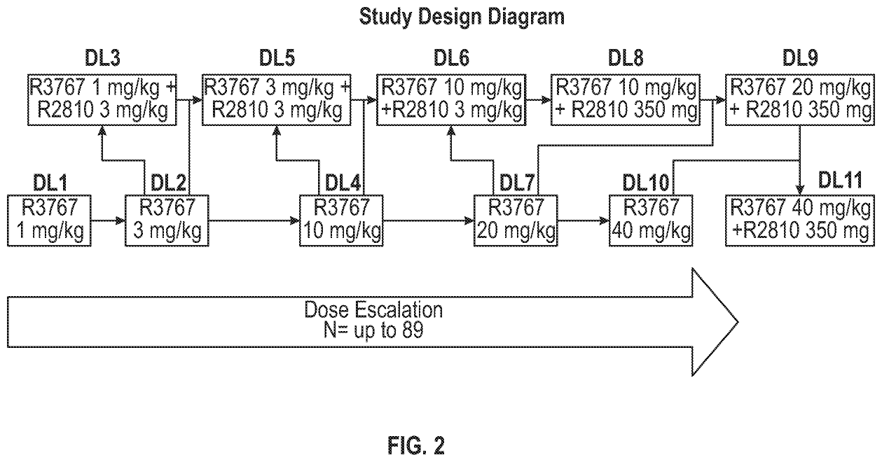

This is a phase 1, first-in-human (FIH), open-label, multicenter, dose escalation study of the safety, tolerability, activity, and pharmacokinetics of REGN3767 administered as monotherapy and in combination with REGN2810 in patients with advanced malignancies. A study flow diagram (for an individual patient) is shown in FIG. 1, and a study design diagram showing the dose escalation scheme and cohorts is shown in FIG. 2.

The exemplary anti-PD-1 antibody used in this example is REGN2810 (also known as cemiplimab, LIBTAYO®). The exemplary anti-LAG2 antibody used in this example is REGN3767.

### Objectives

The primary objective of the study is to assess safety and pharmacokinetics in order to determine the selected dose level(s) for expansion of REGN3767 as a monotherapy and in combination with REGN2810 in patients with advanced malignancies, including lymphoma. The primary objective in the dose expansion phase is to assess preliminary anti-tumor activity of REGN3767 alone and in combination with REGN2810 (separately by cohort) as measured by Objective Response Rate (ORR).

The secondary objectives include: (i) assess preliminary anti-tumor activity of REGN3767 alone and in combination with REGN2810 (separately by cohort) in dose escalation as measured by objective response rate (ORR) based on Response Evaluation Criteria in Solid Tumors (RECIST) 1.1 (solid tumors) or Lugano criteria (lymphoma); (ii) assess preliminary anti-tumor activity of REGN3767 alone and in combination with REGN2810 (separately by cohort) in dose escalation and expansion as measured by ORR based on Immune Response Evaluation Criteria in Solid Tumors (iRECIST), best overall response (BOR), duration of response (DOR), disease control rate, and progression free survival (PFS) based on Response Evaluation Criteria in Solid Tumors (RECIST 1.1), iRECIST, and Lugano criteria; (iii) characterize the safety profile in each expansion cohort as determined in the dose escalation phase; (iv) characterize the pharmacokinetics of REGN3767 as monotherapy and REGN3767 and REGN2810 when given in combination; and (v) assess immunogenicity as measured by anti-drug antibodies (ADA) for REGN3767 and REGN2810.

Additional objectives include: (i) assess overall survival; (ii) assess tumor volume; (iii) assess any relationship of immunogenicity as measured by ADAs to REGN2810 and REGN3767 with drug concentrations; (iv) assess pharmacodynamic changes in putative serum biomarkers (which may include but are not limited to cytokines, circulating tumor nucleic acids, etc.); (v) conduct pharmacokinetics/pharmacodynamics analyses (E-R analyses) on relevant exploratory biomarkers; (vi) assess the predictive potential and correlation to clinical response for biomarkers of interest that may include, but are not limited to: circulating tumor nucleic acids, PBMC subset distribution and expression of immune checkpoint molecules and other biomarkers of interest; tumor RNA expression, number and distribution of tumor infiltrating lymphocytes (CD8+ T-cells, CD4+ T-cells, T-regulatory cells, and tissue permitting, other subtypes such as B-cells, myeloid-derived cells, NK cells, etc), expression levels (messenger RNA and/or protein) of PD-1, PD-L1, LAG-3, MHC class II and possibly other immune modulators or their ligands, mutations in known oncogenes and potential tumor neoantigens, and tumor mutational burden.

### Study Population

The target population for the dose escalation phase comprises patients with advanced malignancies who have not received prior therapy with an anti-LAG-3 drug and who are not candidates for standard therapy or for whom no available therapy is expected to convey clinical benefit, and patients with malignancies that are incurable and have failed to respond to or have shown tumor progression despite standard therapy.

The target population for the expansion cohorts includes patients with select malignancies (see Table 1) who have not received prior therapy with an anti-LAG-3 drug and who:
have not previously received therapy with anti-PD-1/PD-L1 but are appropriate candidates to receive anti-PD-1-based therapy (cohorts 1, 3, 5, 6, 9, and 11), or
have previously tolerated anti-PD-1/PD-L1 based therapy for at least 6 weeks, but subsequently progressed on that therapy or had SD or a PR as best response with subsequent stable response for 6 months (cohorts 2, 4, 7, 10, 12 and 13), or
are not candidates for standard therapy, or for whom no available therapy is expected to convey clinical benefit and are appropriate for REGN3767 monotherapy (cohort 8), or
are appropriate for REGN3767 monotherapy and who do not have access to approved anti-PD-1 therapy for their disease (cohort 14), or
have unresectable or metastatic cutaneous melanoma and are naïve to systemic therapy (cohort 15), or
have not received prior systemic therapy for unresectable or metastatic cutaneous melanoma but have received neoadjuvant or adjuvant therapy (which may include anti-PD-1/PD-L1 therapy) with a treatment-free and disease-free interval of >6 months (cohort 16).

TABLE 1
Expansion Cohorts

| Expansion | | Anti-PD-1/PD-L1 | Anti-PD-1/PD-L1 | |
|---|---|---|---|---|
| Cohort | Tumor Type | Naïve¹ | Experienced² | Treatment |
| 1 | NSCLC | X | | Combination |
| | | | | REGN3767 and |
| | | | | REGN2810 |
| 2 | NSCLC | | X | Combination |
| | | | | REGN3767 and |
| | | | | REGN2810 |
| 3 | ccRCC | X | | Combination |
| | | | | REGN3767 and |
| | | | | REGN2810 |
| 4 | ccRCC | | X | Combination |
| | | | | REGN3767 and |
| | | | | REGN2810 |
| 5 | TNBC | X | | Combination |
| | | | | REGN3767 and |
| | | | | REGN2810 |
| 6 | Melanoma | X | | Combination |
| | | | | REGN3767 and |
| | | | | REGN2810 |
| 7 | Melanoma | | X | Combination |
| | | | | REGN3767 and |
| | | | | REGN2810 |
| 8 | DLBCL | X | | REGN3767 |
| | | | | monotherapy |
| 9 | DLBCL | X | | Combination |
| | | | | REGN3767 and |
| | | | | REGN2810 |
| 10 | DLBCL | | X | Combination |
| | | | | REGN3767 and |
| | | | | REGN2810 |
| 11 | HNSCC | X | | Combination |
| | | | | REGN3767 and |
| | | | | REGN2810 |
| 12 | HNSCC | | X | Combination |
| | | | | REGN3767 and |
| | | | | REGN2810 |
| 13 | CSCC | | X | Combination |
| | | | | REGN3767 and |
| | | | | REGN2810 |
| 14 | CSCC | X | | REGN3767 |
| | | | | monotherapy |
| 15 | Melanoma (systemic | X | | Combination |
| | treatment-naïve) | | | REGN3767 and |
| | | | | REGN2810 |

| | | | |
|---|---|---|---|
| 16 | Melanoma | Patients must have | Combination |
| | (post-neoadjuvant/ | received neoadjuvant | REGN3767 and |
| | adjuvant) | and/or adjuvant therapy. | REGN2810 |
| | | This may include, but | |
| | | is not limited to, | |
| | | anti-PD-1/PD-L1 therapy | |

### Inclusion Criteria

A patient must meet the following criteria to be eligible for inclusion in the study:

1. Men and women 18 years of age.

2. Dose escalation cohorts: Patients with histologically or cytologically confirmed diagnosis of malignancy (including lymphoma) with demonstrated progression of a tumor for whom there is no available therapy likely to convey clinical benefit AND who have not been previously treated with a PD-1/PD-L1 inhibitor. These patients do not require measurable disease per RECIST 1.1 or Lugano criteria.

3. Dose expansion cohorts: Patients with histologically or cytologically confirmed diagnosis of 1 of the following tumors with measurable disease^{†}per RECIST 1.1 or Lugano criteria meeting the following criteria:
Anti-PD-1/PD-L1 naïve stage IIIB, IIIC, or IV NSCLC (non-small cell lung cancer) either without prior therapy for metastatic disease or with disease progression/recurrence after one platinum-containing regimen. Patients with known targetable gene mutations or rearrangements (EGFR mutation, ALK rearrangement, ROS1 rearrangement) are excluded (cohort 1). The decision on whether to perform the test and the test method to be used will follow the requirements of local procedures or guidelines
Anti-PD-1/PD-L1 experienced* stage IIIB, IIIC, or IV NSCLC with no more than 2 prior therapies for metastatic disease. Patients with known targetable gene mutations or rearrangements (EGFR mutation, ALK rearrangement, ROS1 rearrangement) are excluded (cohort 2). The decision on whether to perform the test and the test method to be used will follow the requirements of local procedures or guidelines
Anti-PD-1/PD-L1 naïve advanced or metastatic ccRCC with a clear cell component who had received no more than 2 previous regimens of anti-angiogenic therapy (cohort 3).
Anti-PD-1/PD-L1 experienced* advanced or metastatic ccRCC (clear cell renal cell carcinoma) with a clear cell component who had received no more than 2 previous regimens of anti-angiogenic therapy (cohort 4)
Anti-PD-1/PD-L1 naïve metastatic TNBC (estrogen, progesterone, and human epidermal growth factor receptor 2 negative breast cancer) who have received 5 or fewer prior lines of therapy (cohort 5)
Anti-PD-1/PD-L1 naïve advanced or metastatic non-uveal melanoma^{#} who have received no more than 2 previous regimens for metastatic disease (cohort 6)
Anti-PD-1/PD-L1 experienced*advanced or metastatic non-uveal melanoma^{#} who have received no more than 2 previous regimens for metastatic disease (cohort 7)
Anti-PD-1/PD-L1 naïve relapsed/refractory DLBCL (diffuse large B-cell lymphoma) who have either progressed after or are not candidates for autologous stem cell transplant. The definition includes patients with complex histology that is predominantly DLBCL or high-grade B-cell lymphoma with MYC, BCL2 and/or BCL6 rearrangements (“double or triple hit”) with DLBCL morphology (cohorts 8 and 9) 11
Anti-PD-1/PD-L1 experienced* relapsed/refractory DLBCL who have either progressed after or are not candidates for autologous stem cell transplant. The definition includes patients with complex histology that is predominantly DLBCL or high-grade B-cell lymphoma with MYC, BCL2 and/or BCL6 rearrangements (“double or triple hit”) with DLBCL morphology (cohort 10)
Anti-PD-1/PD-L1 naïve recurrent and/or metastatic HNSCC (irrespective of HPV status) with no curative options who either decline or are ineligible for platinum-based chemotherapy or who have had tumor progression or recurrence within 6 months of last dose of platinum therapy in the adjuvant (i.e., with radiation after surgery), primary (i.e., with radiation), recurrent, or metastatic setting. In addition to radiographic progression, clinical progression is allowed and is defined as progression of a lesion at least 10 mm in size that is amenable to caliper measurement (e.g., superficial skin lesion as per RECIST 1.1) or a lesion that has been visualized and photographically recorded with measurements and shown to have progressed. See inclusion criterion [00154] for additional details (cohort 11)
Anti-PD-1/PD-L1 experienced* recurrent and/or metastatic HNSCC (irrespective of HPV status) with no curative options. See inclusion criterion [00154] for additional details (cohort 12)
Anti-PD-1/PD-L1 experienced* locally advanced or metastatic CSCC (cutaneous squamous cell carcinoma). Patients with locally advanced CSCC must not be appropriate for surgery. Acceptable reasons for surgery to be considered inappropriate are one or both of the following: 1) recurrence of CSCC after surgical procedures and an expectation that curative resection would be unlikely, and/or 2) substantial morbidity or deformity anticipated from surgery. See inclusion criterion [00155] for additional details (cohort 13)
Anti-PD-1/PD-L1 naïve locally advanced or metastatic CSCC. Patients may not have access to an anti-PD-1 therapy approved for the treatment of advanced CSCC. Patients with locally advanced CSCC must not be appropriate for surgery. Acceptable reasons for surgery to be considered inappropriate are one or both of the following: 1) recurrence of CSCC after surgical procedures and an expectation that curative resection would be unlikely, and/or 2) substantial morbidity or deformity anticipated from surgery. See inclusion criterion [00155] for additional details (cohort 14)
Systemic treatment-naïve unresectable or metastatic cutaneous melanoma§. Cohort 6 must be filled prior to enrolling in this cohort (cohort 15)
Unresectable or metastatic cutaneous melanoma§ with prior neoadjuvant and/or adjuvant therapy for melanoma but with no previous systemic therapy for unresectable or metastatic disease (cohort 16).

To be eligible for this cohort, a patient must:
Have never received systemic therapy for unresectable or metastatic disease
Have received prior neoadjuvant and/or adjuvant therapy. Treatment modality may include, but is not limited to: anti-PD-1/anti-PD-L1, anti-CTLA-4, BRAF/MEK inhibitors
Have completed the neoadjuvant and/or adjuvant treatment regimen (i.e., did not discontinue for toxicity)
Have had a treatment-free and disease-free interval of >6 months

### Notes:

^{†}A previously irradiated lesion may be followed as a target lesion as long as progression has been confirmed after radiation therapy. Previously irradiated lesions may be followed as non-target lesions if there is at least 1 other measurable target lesion.

*Anti-PD-1/PD-L1 experienced patients must have had most recent dose of anti-PD-1/PD-L1 not more than 3 months prior to screening (Blasig, 2017).

Anti-PD-1/PD-L1 experienced is defined as having tolerated or tolerating anti-PD-1/PD-L1 therapy for a minimum of 6 weeks (i.e., did not discontinue due to toxicity) and having either: (i) disease progression either on therapy with anti-PD-1/PD-L1 or within 12 weeks of the last dose; or (ii) SD or a PR as best response with subsequent stable response (no greater than 70% decline from baseline) for 6 months while on anti-PD-1/PD-L1 therapy.

*Anti-PD-1/PD-L1 experienced patients should have had an anti-PD-1/PD-L1-containing regimen lasting at least 6 weeks to be considered experienced. If patients had received anti-PD-1/PD-L1 for less than 6 weeks and did not discontinue due to toxicity and/or on-treatment imaging showing disease progression, they may be considered anti-PD-1/PD-L1 naïve for enrollment.

#Acral lentiginous and mucosal melanomas combined will be limited to <15% of each cohort.

§ Acral lentiginous melanoma will be limited to <15% of each cohort

§ Patients in cohort 9 must have received 89Zr-DFO-REGN3767 (anti-LAG-3 immuno-PET [iPET] antibody) prior to enrolling in the study

4. Eastern Cooperative Oncology Group performance status of 0 or 1

5. Estimated life expectancy of at least 3 months

6. Adequate organ and bone marrow function as follows:
Hemoglobin ≥9.0 g/dL
Absolute neutrophil count ≥1.5×10⁹/L
Platelet count ≥75×10⁹/L (≥50×10⁹/L if lymphoma patient)
Serum creatinine ≤1.5× upper limit of normal (ULN) or estimated glomerular filtration rate >50 mL/min/1.73 m²(dose escalation cohorts) or estimated glomerular filtration rate >30 mL/min/1.73 m² (dose expansion cohorts)
Total bilirubin ≤1.5×ULN (Note: For patients with known Gilbert's
Syndrome, x the institutional ULN is permitted)
Aspartate aminotransferase and alanine aminotransferase (ALT) ≤3×ULN (or ≤5×ULN, if liver metastases)
Alkaline phosphatase ≤2.5×ULN (or ≤5.0×ULN, if liver or bone metastases)
Calcium ≤12.5 mg/dL. Patients who have a history of hypercalcemia controlled on bisphosphonate therapy with a decrease to ≤12.5 mg/dL at the time of screening are eligible.

7. Willing and able to comply with clinic visits and study-related procedures

8. Provide signed informed consent

9. For patients with HNSCC, patients will have primary tumor sites of oral cavity, oropharynx, larynx, or hypopharynx. Squamous cell carcinoma of lymph node in cervical chain of the neck, with unknown primary, is an eligible diagnosis if HPV-associated (by p16 IHC or HPV in situ hybridization and/or HPV polymerase chain reaction [PCR]). Patients with nasopharyngeal carcinoma are excluded.

10. For patients with CSCC: Histologically confirmed diagnosis of invasive CSCC

Notes on tumor primary site: The intent is to study patients whose tumors are likely to be due to UV exposure. Patients for whom the primary site of squamous cell carcinoma was the dry red lip (vermillion) are not eligible. Patients with tumors arising on the cutaneous hair-bearing (non-glabrous) lip with extension onto dry red lip (vermillion) may be eligible after communication with and approval from medical monitor. Patients for whom the primary site of squamous cell carcinoma was the anogenital area (penis, scrotum, and perianal region) are not eligible. Patients for whom the primary site is nose are only eligible if it can be established unambiguously that the primary site was skin, not nasal mucosa with outward extension to skin.

Notes on tumor histology: Patients with mixed histology (e.g., sarcomatoid, adenosquamous) generally will not be eligible. Patients with mixed histology in which the predominant histology is invasive CSCC (with only a minimal component of mixed histology) may be eligible, after communication with and approval from medical monitor.

### Exclusion Criteria

A patient who meets any of the following criteria will be excluded from the study:

1. Currently receiving treatment in another study, has participated in a study of an investigational agent and received treatment, has used an investigational device within 4 weeks of first dose of study therapy (except 89Zr-DFO-REGN3767), has received treatment with an approved systemic therapy within 3 weeks of first dose of study therapy, or has received any previous systemic therapy within 5 half-lives of first dose of study therapy (whichever is longer). Patients previously treated with bevacizumab, cetuximab, rituximab, or other non-immunomodulatory antibodies with half-lives longer than 7 days are permitted if at least 30 days have elapsed since last treatment. Patients previously treated with immunomodulatory antibodies with half-lives longer than 7 days, such as ipilimumab, are permitted if at least 3 half-lives have elapsed since last treatment. Patients previously treated with immunomodulatory cellular therapies such as CAR-T cells, are permitted if at least 30 days have elapsed since last treatment.

NOTE: For anti-PD-1/PD-L1 experienced patients enrolled in appropriate cohorts: washout periods noted for systemic therapy above do not apply to prior anti-PD-1/PD-L1 therapy. Prior anti-PD-1/PD-L1 therapy must have occurred more than 3 weeks prior to first dose of study drug, regardless of half-life or approval status of the drug. Prior anti-PD-1/PD-L1 therapy must have occurred less than 3 months prior to screening. Patients who have received 89Zr-DFO-REGN3767 (anti-LAG-3 immuno-PET [iPET] antibody) are permitted in expansion cohort 9 regardless of the time since administration. Patients previously treated with bevacizumab, cetuximab, rituximab, or other non-immunomodulatory antibodies with half-lives longer than 7 days are permitted after a discussion with the sponsor if at least 30 days have elapsed since last treatment. Patients previously treated with immunomodulatory antibodies with half-lives longer than 7 days, such as ipilimumab, are permitted after discussion with the sponsor if at least 3 half-lives have elapsed since last treatment. Patients previously treated with cellular therapies such as CAR-T cells are permitted after agreement with the sponsor if at least 30 days have elapsed since last treatment.

2. Prior treatment with any LAG-3-targeting biologic or small molecule, with the exception of 89Zr-DFO-REGN3767 (anti-LAG-3 iPET antibody).

3. Radiation therapy within 2 weeks prior to enrollment and not recovered to baseline from any AE due to radiation.

4. Expansion cohorts only: Another malignancy that is progressing or requires active treatment with the exception of non-melanoma skin cancer that has undergone potentially curative therapy or in situ cervical carcinoma, or any other tumor that has been deemed to be effectively treated with definitive local control for at least 2 years prior to enrollment.

5. Primary brain tumors and untreated or active central nervous system metastases. Patients with previously treated central nervous system metastases may participate provided they are stable (i.e., without evidence of progression by imaging for at least 6 weeks prior to the first dose of study treatment, and any neurologic symptoms have returned to baseline), and there is no evidence of new or enlarging central nervous system metastases, and the patient does not require any systemic corticosteroids for management of central nervous system metastases within 4 weeks prior to the first dose of REGN3767/REGN2810.

6. Encephalitis, meningitis, or uncontrolled seizures in the year prior to informed consent.

7. Ongoing or recent (within 5 years) evidence of significant autoimmune disease that required treatment with systemic immunosuppressive treatments, which may suggest risk for irAEs. The following are not exclusionary: vitiligo, childhood asthma that has resolved, hypothyroidism that required only hormone replacement, type 1 diabetes, or psoriasis that does not require systemic treatment.

8. Corticosteroid therapy (>10 mg prednisone/day or equivalent) within 1 week prior to the first dose of study drug. Patients who require a brief course of steroids are not excluded.

9. Known history of, or any evidence of interstitial lung disease, or active, non-infectious pneumonitis (past 5 years). A history of radiation pneumonitis in a previous radiation field is permitted.

10. Uncontrolled infection with human immunodeficiency virus, hepatitis B or hepatitis C infection; or diagnosis of immunodeficiency that is related to, or results in chronic infection. Mild cancer-related immunodeficiency (such as immunodeficiency treated with gamma globulin and without chronic or recurrent infection) is allowed.

### Notes:

Patients will be tested for HIV, HCV, and HBV at screening.

Patients with HIV who have controlled infection (undetectable viral load and CD4 count above 350 either spontaneously or on a stable antiviral regimen) are permitted. For patients with controlled HIV infection, monitoring will be performed per local standards.

Patients with hepatitis B (HepBsAg+) who have controlled infection (serum hepatitis B virus DNA PCR that is below the limit of detection AND receiving anti-viral therapy for hepatitis B) are permitted. Patients with controlled infections must undergo periodic monitoring of HBV DNA. Patients must remain on anti-viral therapy for at least 6 months beyond the last dose of investigational study drug.

Patients who are hepatitis C virus antibody positive (HCV Ab+) who have controlled infection (undetectable HCV RNA by PCR either spontaneously or in response to a successful prior course of anti-HCV therapy) are permitted.

Patients with HIV or hepatitis must have their disease reviewed by the specialist (e.g., infectious disease or hepatologist) managing this disease prior to commencing and throughout the duration of their participation in the trial.

11. Active infection requiring systemic therapy.

12. Receipt of a live vaccine within 30 days of planned start of study medication.

13. Major surgical procedure, open biopsy, or significant traumatic injury within 4 weeks prior to screening.

14. Myocardial infarction within 6 months prior to the first dose of study therapy.

15. Prior allogeneic stem cell transplant or solid organ transplant.

16. Any medical condition that would make participation in the study not in the best interest of the patient.

17. Patients with treatment-related immune-mediated AEs of grade 2 or higher that either required permanent discontinuation of patient's most recent anti-PD-1/PD-L1 or anti-CTLA-4 therapy or that have not resolved to baseline at least 30 days prior to initiation of study therapy.

NOTE: Endocrine immune-mediated AEs controlled with hormonal or other non-immunosuppressive therapies (without resolution) or grade 1 irAEs affecting any organ system with resolution prior to enrollment are allowed.

18. [Exclusion criterion removed]

19. Known psychiatric or substance abuse disorders that would interfere with participation with the requirements of the study.

20. Women who are pregnant, breastfeeding or expecting to conceive or men planning to father a child within the projected duration of the study (screening visit through 180 days after the last dose of study drug).

21. Sexually active men* or women of childbearing potential** who are unwilling to practice highly effective contraception prior to the start of the first treatment, during the study, and for at least 6 months after the last dose of study drug is administered. Adequate contraceptive measures include stable use of oral contraceptives such as combined estrogen and progestogen and progestogen only hormonal contraception or other prescription pharmaceutical contraceptives for 2 or more menstrual cycles prior to screening; intrauterine device; intrauterine hormone-releasing system; bilateral tubal ligation; vasectomy and sexual abstinence.***

*Contraception is not required for men with documented vasectomy.

**Postmenopausal women must be amenorrheic for at least 12 months in order not to be considered of childbearing potential. Pregnancy testing and contraception are not required for women with documented hysterectomy or tubal ligation.

***Sexual abstinence is considered a highly effective method only if defined as refraining from heterosexual intercourse during the entire period of risk associated with the study treatments. The reliability of sexual abstinence needs to be evaluated in relation to the duration of the clinical study, and the preferred and usual lifestyle of the patient.

22. Member of the clinical site study team and/or his or her immediate family

23. [Exclusion criterion removed]

### Study Design

This is a phase 1, first-in-human, open-label, multicenter, dose-escalation study of the safety, tolerability, activity, and pharmacokinetics of REGN3767 administered as monotherapy and in combination with REGN2810 in patients with advanced malignancies. Eleven dose escalation cohorts are planned with REGN3767 as monotherapy and as combination therapy with REGN2810. A study flow diagram (for an individual patient) is shown in FIG. 1, and a study design diagram showing the dose escalation scheme and cohorts is shown in FIG. 2.

### Duration

After a screening period of up to 28 days, patients receive up to seventeen 21-day treatment cycles (for a total of up to 51 weeks of treatment), followed by a 24-week follow-up period. Each patient receives REGN3767 (+/−REGN2810) every 21 days. The total study duration is approximately 78 weeks (546 days), excluding the screening period. However, an additional 51 weeks of treatment is available to patients who continue to show benefit.

Treatment will continue until the 51-week treatment period is complete, or until disease progression, unacceptable toxicity, withdrawal of consent, or study withdrawal criterion is met. After completion of 51 weeks of therapy, therapy may be continued for an additional 51 weeks.

Response assessment is performed every 6 weeks for the first 24 weeks, then every 9 weeks for the subsequent 27 weeks, regardless of delays in dosing of study drugs. After a minimum of 24 weeks of treatment (minimum 8 treatment cycles), patients with confirmed complete response (CR) may elect to discontinue treatment and continue with all relevant study assessments. Similarly, patients who have been treated for a minimum of 24 weeks with stable disease (SD) or partial response (PR) that has been maintained for 3 successive tumor evaluations may elect to discontinue treatment but continue with all relevant study assessments.

Patients who progress within 6 months after stopping for CR or stable PR or SD (including completion of study therapy), are allowed to resume study treatment (following confirmation of relevant study eligibility criteria) at the same dose (at Resumption Cycle 1) or dose chosen for expansion (whichever is higher). Patients can receive up to 17 additional cycles of therapy. If patients are resuming the same dose, pharmacokinetics and biomarker samples are collected as per the Resumption of Treatment Schedule of Events. If patients start treatment at a different dose or combination than the one at which they were originally treated, samples and safety assessments are requested and patients start according to the Retreatment Schedule of Events.

For patients who experience a response and subsequently progress, a tumor biopsy at the time of progression is requested.

Patients in dose escalation who tolerate a minimum of 2 doses of REGN3767 monotherapy, and patients in expansion cohorts who tolerate a minimum of 1 dose of REGN3767 monotherapy, but who subsequently demonstrate progressive disease (PD), have the option of entering the re-treatment phase and adding REGN2810 350 mg to the highest dose level or fixed dose equivalent of REGN3767 tolerated in combination with REGN2810 up to that point (if one is known) in an attempt to “rescue” a response by combined LAG-3 and PD-1 blockade. The selection of REGN2810 dose is based on the dose levels known to be tolerable in combination with REGN3767 at the time the patient transitions from monotherapy to combination re-treatment to receive “rescue” therapy. In expansion cohorts, patients receiving monotherapy who tolerate a minimum of 1 dose of REGN3767 have the option to receive the combination therapy dose chosen for expansion (350 mg cemiplimab+1600 mg REGN3767). Patients enter “rescue combination cycle 1” and receive up to 17 cycles of combination therapy. If there is a difference between the patient's current dose of REGN3767 monotherapy and the known tolerable dose of REGN3767 in combination with REGN2810, an appropriate time is determined to start administration of each drug in combination therapy to allow drug concentrations of REGN3767 in the blood to decline to levels that are tolerable in combination. Patients should continue with scheduled visits. Pharmacokinetics and biomarker samples are collected according to the Rescue Schedule of Events. Incorporation of safety data for these patients into the dose evaluation (escalation) phase is described below.

Patients enter follow-up after completing the treatment period of up to 51 weeks (or, if patients enter continuation or rescue, at the end of the additional 51 weeks of treatment). Patients who resume treatment or receive retreatment should also undergo follow-up procedures after stopping each respective treatment. Patients may enter follow-up more than once.

Safety evaluations are conducted at each study drug dosing visit.

Eleven dose escalation cohorts are planned. Five dose levels of REGN3767 (1, 3, 10, 20 and 40 mg/kg) are investigated as monotherapy. Three dose levels of REGN3767 (1, 3, and 10 mg/kg) are investigated in combination with REGN2810 at 3 mg/kg. The REGN3767 10 mg/kg combined with REGN2810 3 mg/kg was determined to be tolerable, and subsequent dose levels of REGN3767 (10, 20 and 40 mg/kg, or an equivalent fixed dose) are investigated in combination with REGN2810. Since both weight-based (in mg/kg units) and fixed dosing (in mg units) are similar with regards to reducing pharmacokinetic inter-individual variability across a wide range of monoclonal antibody therapies tested, REGN3767 is dosed in combination with a 350 mg fixed dose of cemiplimab in dose escalation. Since the MTD of REGN3767 has not been reached, the 2 additional cohorts with 40 mg/kg REGN3767 (monotherapy and combination with 350 mg cemiplimab) are investigated. The first cohort to be enrolled receives REGN3767 monotherapy at 1 mg/kg. Subsequent enrollment of each additional cohort may be limited by the number of dose limiting toxicities (DLT) observed in prior cohorts.

Enrollment in the expansion cohorts begins once a range of tolerable dose levels are identified in the dose escalation. More than one dose level may be studied in expansion cohorts.

Additional expansion cohorts may be enrolled after confirmation of the selected dose(s). Solid tumor expansion cohorts have a Simon 2-stage design, and DLBCL expansion cohorts have a single (pilot) stage. A patient is assigned to a specific treatment cohort based on the patient's tumor type; the presence or absence of prior anti-PD-1/anti-programmed death ligand 1 (PD-L1) therapy; the assessment of the appropriateness of a therapy regimen for that patient; and the availability of patient slots in the assigned treatment cohort. If safety issues develop in an individual expansion cohort during stage 1 of the Simon 2-stage design or the lymphoma pilot cohorts, enrollment may be paused. Enrollment may be resumed at the same or lower dose of REGN3767. Safety issues triggering a pause could include early or late safety events. Patients are treated with 20 mg/kg Q3W REGN3767 monotherapy (dose determined from monotherapy dose escalation findings), or the fixed dose equivalent, or combination of REGN3767 and REGN2810 (dose level[s] to be determined by combination dose-escalation findings) for up to 51 weeks. The enrollment of stage 2 for each solid tumor expansion cohort will occur only if the minimum number of tumor responses is observed at stage 1.

Both weight-based doses and fixed doses for REGN3767 and REGN2810 are tested in this study. The weight-based dosing may be converted to a suitable fixed dose, since both fixed dosing and weight-based dosing have been shown to perform similarly in reducing pharmacokinetic variability. Therefore, the study design will result in the selection of a fixed dose combination of REGN3767 and REGN2810.

For patients in the expansion cohorts, a pretreatment biopsy is required (unless the patient had a recent biopsy from another study), and an on-treatment tumor biopsy on day 29±7 is required, unless the patient is clinically unstable or otherwise unable to tolerate this procedure. In the event an on-treatment (day 29) biopsy is not medically appropriate, a discussion with the medical monitor must take place prior to day 29 to explain and document the rationale.

### Study Cohorts

### Dose Escalation Cohorts

Eleven dose escalation cohorts are planned (FIG. 2). Five dose levels of REGN3767 (1, 3, 10, 20, and 40 mg/kg) are investigated as monotherapy. Three dose levels of REGN3767 (1, 3, and 10 mg/kg) are investigated in combination with REGN2810 at 3 mg/kg. If the REGN3767 10 mg/kg combined with REGN2810 3 mg/kg is determined to be tolerable, subsequent dose levels of REGN3767 (10, 20, and 40 mg/kg) are investigated in combination with REGN2810. Since both weight-based (in mg/kg units) and fixed dosing (in mg units) are similar with regards to reducing pharmacokinetic inter-individual variability across a wide range of monoclonal antibody therapies tested, REGN3767 is dosed in combination with a 350 mg fixed dose of REGN2810 in dose escalation. 2 additional cohorts with 40 mg/kg REGN3767 (or an equivalent fixed dose) (monotherapy and combination with 350 mg REGN2810) are investigated.

### Expansion Cohorts

Disease specific cohorts are enrolled after selection of the dose level(s) for expansion. The timing and order of opening enrollment of the expansion cohorts is determined based on available data. Cohorts may open at different times during the study. Solid tumor expansion cohorts have a Simon 2-stage design and DLBCL expansion cohorts will have a single (pilot) stage. A patient is assigned to a specific treatment cohort based on the patient's tumor type, presence, or absence of prior anti-PD-1/anti-PD-L1 therapy, the assessment of the appropriateness of a therapy regimen for that patient, and the availability of patient slots in the assigned treatment cohort. If safety issues develop in an individual expansion cohort during stage 1 of the Simon 2-stage design or the lymphoma pilot cohorts, enrollment may be paused. Enrollment may be resumed at the same or lower dose of REGN3767 and/or REGN2810. Safety issues triggering a pause could include early or late safety events. Patients are treated with 20 mg/kg REGN3767 (dose selected from monotherapy dose escalation findings) monotherapy, or combination of REGN3767 and REGN2810 (doses determined by combination dose-escalation findings) for up to 51 weeks. Weight-based dosing for REGN3767 and REGN2810 may be converted to a suitable fixed dose, since both fixed and weight-based dosing are similar with regards to reducing pharmacokinetic inter-individual variability across a wide range of monoclonal antibody therapies tested (Wang, 2009). For fixed dose cohorts, patients are treated with 1600 mg of REGN3767 monotherapy, or a combination of REGN3767 and REGN2810 (1600 mg of REGN3767 and 350 mg of REGN2810). The enrollment of stage 2 for each solid tumor expansion cohort occurs if the minimum number of tumor responses is observed at stage 1.

Weight-based dosing for REGN3767 (in mg/kg units) and REGN2810 is converted to suitable fixed doses (in mg units) for most expansion cohorts, with the exception of expansion cohort 8, since both weight-based and fixed dosing are similar with regards to reducing pharmacokinetic inter-individual variability across a wide range of monoclonal antibody therapies tested.

Patients in monotherapy expansion cohorts 8 and 14 are treated with REGN3767 20 mg/kg and at the fixed-dose equivalent of 1600 mg (converted from 20 mg/kg REGN3767 for an assumed patient body weight of 80 kg), respectively, for up to 51 weeks. This weight-based dose and fixed-dose equivalent have been selected based on information collected during dose escalation as well as from the monotherapy weight-based experience in expansion cohort 8 (for fixed dosing in cohort 14). In the dose escalation phase, the MTD for REGN3767 has not been reached, and there were no safety concerns for REGN3767 20 mg/kg monotherapy or for the combination of 20 mg/kg REGN3767 plus 350 mg REGN2810. Furthermore, in expansion cohort 8, REGN3767 monotherapy activity has been observed with 20 mg/kg Q3W. Therefore, new monotherapy cohorts in the dose expansion (e.g., cohort 14) are opened at the fixed dose equivalent of 1600 mg REGN3767. See Table 2.

If stage 2 for a given cohort meets the criteria for success, then additional patients may be enrolled in the cohort up to a maximum of 40 patients for solid tumor cohorts. For DLBCL, cohort size may be increased to 20 patients if the cohort is successful. For DLBCL expansion cohort 9, 20 patients are enrolled to match the enrollment requirements of an anti-LAG-3 iPET study. In addition, if the characteristics of a given enrolled cohort vary from the expected patient population based on baseline clinical data (e.g., tumor subtype) or emerging biomarker data (e.g., tumor PD-L1 and/or LAG-3 expression, etc.) such that the true response rate is higher than the observed response rate, additional cohorts may be opened in that tumor type with more defined selection criteria.

TABLE 2
A List of Expansion Cohorts and Their Respective Doses

| Expansion | | Anti-PD-1/PD-L1 | | | |
|---|---|---|---|---|---|
| Cohort | Tumor Type | Naïve/Experienced | | Treatment | Treatment Regimen |
| 1 | NSCLC | Naive | | Combination | 1600 mg REGN3767+ |
| | | | | | 350 mg REGN2810 |
| 2 | NSCLC | | Experienced | Combination | 1600 mg REGN3767+ |
| | | | | | 350 mg REGN2810 |
| 3 | ccRCC | Naive | | Combination | 1600 mg REGN3767+ |
| | | | | | 350 mg REGN2810 |
| 4 | ccRCC | | Experienced | Combination | 1600 mg REGN3767+ |
| | | | | | 350 mg REGN2810 |
| 5 | TNBC | Naive | | Combination | 1600 mg REGN3767+ |
| | | | | | 350 mg REGN2810 |
| 6 | Melanoma | Naive | | Combination | 1600 mg REGN3767+ |
| | | | | | 350 mg REGN2810 |
| 7 | Melanoma | | Experienced | Combination | 1600 mg REGN3767+ |
| | | | | | 350 mg REGN2810 |
| 8 | DLBCL | Naive | | REGN3767 | 20 mg/kg REGN3767 |
| | | | | monotherapy | |
| 9 | DLBCL | Naive | | Combination | 1600 mg REGN3767+ |
| | | | | | 350 mg REGN2810 |
| 10 | DLBCL | | Experienced | Combination | 1600 mg REGN3767+ |
| | | | | | 350 mg REGN2810 |
| 11 | Head and Neck | Naive | | Combination | 1600 mg REGN3767+ |
| | | | | | 350 mg REGN2810 |
| 12 | Head and Neck | | Experienced | Combination | 1600 mg REGN3767+ |
| | | | | | 350 mg REGN2810 |
| 13 | CSCC | | Experienced | Combination | 1600 mg REGN3767+ |
| | | | | | 350 mg REGN2810 |
| 14 | CSCC | Naïve | | REGN3767 | 1600 mg REGN3767 |
| | | | | monotherapy | |
| 15 | Melanoma (systemic | Naïve | | Combination | 1600 mg REGN3767+ |
| | treatment-naïve) | | | | 350 mg REGN2810 |

| | | | | |
|---|---|---|---|---|
| 16 | Melanoma | Patients must have | Combination | 1600 mg REGN3767+ |
| | (post-neoadjuvant/ | received neoadjuvant | | 350 mg REGN2810 |
| | adjuvant) | and/or adjuvant therapy. | | |
| | | This may include, but | | |
| | | is not limited to, | | |
| | | anti-PD-1/PD-L1 therapy | | |

### Dose Escalation Rules

A modification of the traditional 3+3 design (“4+3”) was used for evaluation of all dose levels for both REGN3767 monotherapy and REGN3767+REGN2810 cohorts. A diagram of the study design is shown in FIG. 2.

The DLT evaluation period is 28 days. Although a minimum of 3 patients at each dose level is required to be evaluable for DLT, to maximize the efficiency of the phase 1 dose escalation while maintaining patient safety, 4 patients are enrolled at each dose level in case a patient discontinues prior to being evaluable for DLT. The rules for tolerability are as follows:
Tolerability of a dose level is considered achieved if all potentially DLT-evaluable patients complete the 28-day DLT period without a DLT (0 in 3 patients or 0 in 4 patients).
If 3 patients complete the DLT period without experiencing a DLT, but there is a fourth patient in the DLT evaluation period, tolerability of the dose level is only considered achieved when the fourth patient completes the DLT evaluation period or discontinues therapy prior to being evaluable for DLT.
If there is 1 DLT in either 3 or 4 DLT-evaluable patients, then 4 or 3 more patients, respectively, are enrolled, for a total of 7 patients. A dose is considered tolerable if there is 1 DLT in 6 patients or 1 DLT in 7 patients. The MTD is reached if there are 2 or more DLTs in 2 to 7 evaluable patients.
At the highest dose level tolerated, to further evaluate safety, additional 3 to 4 patients may be enrolled for a total of 6 to 10 DLT-evaluable patients. The dose is considered acceptable if there is 0 to 1 DLT in 6 to 8 patients, or 2 DLTs in 9 to 10 patients.

To further evaluate safety, 3 to 4 additional patients may be enrolled at any dose level. These additional patients treated in combination therapy cohorts might have previously received treatment with anti-PD-1 or anti-PD-L1 therapies, though they must meet all other eligibility criteria.

Dose escalation (FIG. 2) will proceed as follows:
If 1.0 mg/kg REGN3767 (DL1) is deemed tolerable (after 3 to 7 patients), enrollment begins at 3.0 mg/kg REGN3767 (DL2).
Following enrollment of 4 patients in DL2, enrollment in the first combination cohort (1.0 mg/kg REGN3767+3.0 mg/kg REGN2810; DL3) may begin.
Dose escalation to 10 mg/kg REGN3767 (DL4) may begin once 3.0 mg/kg REGN3767 (DL2) is deemed tolerable.
The 3.0 mg/kg REGN3767+3.0 mg/kg REGN2810 combination cohort (DL5) only enrolls after both DL2 and DL3 are deemed tolerable.
The 10 mg/kg REGN3767+3.0 mg/kg REGN2810 combination cohort (DL6) only enrolls after both DL4 and DL5 are deemed tolerable.
Dose escalation to 20 mg/kg REGN3767 (DL7) may begin once 10 mg/kg REGN3767 (DL4) is deemed tolerable.

Once a combination MTD or selected dose is identified in the 3 combination cohorts with 3 mg/kg REGN2810 (DL3, DL5 and DL6), enrollment may begin at the identified dose of REGN3767 combined with 350 mg (fixed dose) REGN2810 (DL8). Subsequent combination cohorts also include 350 mg REGN2810, as follows:
The 20 mg/kg REGN3767+350 mg REGN2810 combination cohort (DL9) only enrolls after both DL8 and DL7 are deemed tolerable.
Based on the totality of data, the dose levels selected to move to expansion may be determined after DL9 is enrolled.
Dose escalation to 40 mg/kg REGN3767 (DL10) may begin once 20 mg/kg REGN3767 (DL7) is deemed tolerable.
40 mg/kg REGN3767+350 mg REGN2810 combination cohort (DL11) only enrolls after DL9 and DL10 are deemed tolerable.
If multiple cohorts are open simultaneously, priority should be given to the lower number cohort.

No intrapatient dose escalation is permitted in the study (except in the setting of retreatment or rescue). Escalation to the next cohort occurs once all of the initial patients enrolled in a cohort (although screening for the next dose cohort may begin prior to confirmation that the current dose is tolerable) have completed the dose-limiting toxicity observation period and the data have been reviewed.

In the first monotherapy cohort, 48 hours are required between first study drug administration for the first 4 patients enrolled (i.e., in DL1). For example, if patient #1 is treated on Monday, patient #2 cannot be treated before Wednesday. If no unexpected toxicity is observed, each subsequent monotherapy cohort can enroll patients without implementing a waiting period. This same approach (48-hour waiting period) is utilized with initial enrollment in the first combination cohort (i.e., in DL3).

### Dose-Limiting Toxicities

The DLT observation period for determination of safety for dose escalation or initiation of new combination therapy is defined as 28 days starting with cycle 1, day 1, with the intent to monitor the safety and tolerability of the first 2 doses of study drug(s) (REGN3767 with or without REGN2810 as applicable). To be evaluable for a DLT, a patient must have received at least the first 2 doses of study drug(s) (i.e., day 1 and day 22) and be monitored for at least 28 days following the first administration, and at least 7 days from the second administration or experienced a DLT (defined below) prior to the completion of the DLT period. Delays in the administration of the second dose of study drug(s) beyond day 35 and/or study drug discontinuation are considered a DLT if study-drug-related. The duration of the DLT observation period is therefore longer for patients whose second dose is delayed, and for patients experiencing an AE for which the duration must be assessed in order to determine if the event was a DLT.

In addition to the inability to administer (due to study drug toxicity) dose #2 within the window, a DLT in general is defined as any of the following study drug-related toxicities:

Non-Hematologic Toxicity:
Grade ≥2 uveitis
Any grade non-hematologic toxicity (excluding clinically insignificant laboratory abnormalities such as asymptomatic elevations in amylase or lipase)

Hematologic Toxicity:
Grade 4 neutropenia lasting >7 days
Grade 4 thrombocytopenia
Grade 3 thrombocytopenia with bleeding
Grade ≥3 febrile neutropenia or grade ≥3 neutropenia with documented infection

The frequency, time to onset, and severity of toxicities, as well as the success of standard medical management and dosing interruptions/delays is analyzed to determine if a given toxicity should be considered a DLT for dose escalation purposes. Both irAEs and non-irAEs that meet the definition of a DLT are considered to be DLTs.

In general, any AE is treated as unexpected. Such TEAEs are monitored on an ongoing basis to assess possible differences in event frequency or severity from that observed with other agents that block LAG-3, anti-PD-1/PD-L1 or the combination when data on anti-LAG-3 antibodies (alone or in combination) become publicly available.

Treatment-emergent adverse events (TEAEs) that appear to meet the DLT definition are discussed and the final decision of whether or not the AE meets the DLT definition is based on a careful review of all relevant data and consensus

Regardless of whether a patient remains on study treatment and/or continues to participate in study procedures, such an event counts as a DLT for the involved cohort if the event occurs during the DLT observation period.

### Maximum Tolerated Dose

The MTD is defined as the dose level immediately below that at which dosing is stopped due to 2 or more DLTs out of 6 to 7 evaluable patients, and is determined separately for monotherapy and combination therapy. However, due to the known occurrence of AEs due to monotherapy with REGN2810 and other PD-1/PD-L1 antibodies, for the combination cohorts the intensity, frequency and novelty of combination toxicity may be considered in the determination of MTD and the decision to add additional patients at a dose level. If dose escalation is not stopped for DLTs, it is considered that the MTD has not been determined. An additional 3 patients enroll in each of the monotherapy and combination cohorts deemed the highest dose levels tolerated (i.e., 6 to 10 patients in each of these cohorts). If dose escalation for REGN3767 monotherapy or combination therapy with REGN2810 is stopped at 1.0 mg/kg due to DLTs, a cohort is enrolled at a dose of 0.3 mg/kg. If dose escalation for REGN3767 monotherapy or combination therapy is stopped due to DLTs at the 3.0, 10, or 20 mg/kg dose level, the dose of REGN3767 is reduced to the previously tested dose level for newly enrolled patients (in monotherapy or combination therapy cohorts respectively). No patients are allowed to initiate combination therapy with a dose of REGN3767 that was not tolerable as monotherapy.

### Additional Study Treatment

An additional 51 weeks of study treatment may be provided after the initial 51 weeks of treatment for the following patients:
Patients who develop PD while on study subsequent to having stopped therapy due to CR or prolonged PR/SD after 24 weeks of treatment
Patients who develop PD while in follow-up after having completed at ≥51 weeks/17 cycles of treatment
Patients who develop PD during monotherapy treatment and transition to combination therapy
Patients who decide to continue treatment after having completed 51 weeks/17 cycles of treatment without progressive disease.

### Dose Levels Selected for Expansion or Recommended Phase 2 Dose

The dose level(s) selected for expansion is no higher than the MTD or highest dose tested and may be different for monotherapy and combination therapy cohorts. The determination of the dose level(s) selected for expansion is based on safety and PK data.

Addition of REGN2810 to REGN3767 for Patients with Progressive Disease

Patients in monotherapy cohorts who tolerate a minimum of 2 doses of REGN3767 in dose escalation, and patients who tolerate a minimum of 1 dose of REGN3767 in expansion cohorts, but who subsequently demonstrate PD, have the option of adding 3.0 mg/kg or 350 mg REGN2810 to the highest dose of REGN3767 tolerated in combination with REGN2810 up to that point (if one is known) in an attempt to “rescue” a response by combined LAG-3 and PD-1 blockade. Once a combination REGN3767 dose level with 350 mg REGN2810 is found to be tolerable, patients transitioning to combination (“rescue”) therapy receive combination REGN3767 with REGN2810 350 mg. If there is a difference between the patient's current dose of REGN3767 monotherapy and the known tolerable dose of REGN3767 in combination with REGN2810, an appropriate time is determined to start administration of each drug in combination therapy to allow drug concentrations of REGN3767 in the blood to decline to levels that are tolerable in combination. The appropriate time to administer study drug is based on a linear PK model, assuming a conservative half-life for REGN3767 of 21 days, or the most current available half-life. Patients should continue with scheduled visits. Patients can then receive up to 17 additional cycles of therapy. Since patients start treatment at a combination dose, additional samples and safety assessments are requested. Patients' treatment and assessments follow the Rescue Schedule of Events. These patients do not contribute to the first 3 to 4 in a combination cohort needed for DLT evaluation. However, their safety data is evaluated in the determination of MTD. Patients with prior idelalisib therapy are not eligible for rescue with REGN2810.

### Treatments and Assessments

### Monotherapy Treatment Schedule

REGN3767 is administered in an outpatient setting by IV infusion. Longer infusion durations than those specified below for each cohort are acceptable if interruption is required. Planned monotherapy regimens include:
DL1: 1.0 mg/kg REGN3767 IV infusion over 30 minutes every 21 days for 51 weeks
DL2: 3.0 mg/kg REGN3767 IV infusion over 30 minutes every 21 days for 51 weeks
DL4: 10 mg/kg REGN3767 IV infusion over 30 minutes every 21 days for 51 weeks
DL7: 20 mg/kg REGN3767 IV infusion over 30 minutes every 21 days for 51 weeks
DL10: 40 mg/kg REGN3767 IV infusion over 60 minutes every 21 days for 51 weeks
DL-1m: 0.3 mg/kg REGN3767 IV infusion over 30 minutes every 21 days for 51 weeks (if necessary)
Expansion cohort 8: 20 mg/kg REGN3767 IV infusion over 30 minutes every 21 days for 51 weeks
Expansion cohort 14: 1600 mg REGN3767 IV infusion over 30 minutes every 21 days for 51 weeks

### Combination Treatment Schedule

For combination therapy, the sequence of study drug administration is REGN3767 first followed by REGN2810 on the same day. Study drugs are administered in an outpatient setting by IV infusion. Longer infusion durations than those specified below for each cohort are acceptable if interruption is required. Planned combination regimens to be assigned include:
DL3: 1.0 mg/kg REGN3767 and 3.0 mg/kg REGN2810 IV infusion over 30 minutes each, every 21 days for 51 weeks
DL5: 3.0 mg/kg REGN3767 and 3.0 mg/kg REGN2810 IV infusion over 30 minutes each, every 21 days for 51 weeks
DL6: 10 mg/kg REGN3767 and 3.0 mg/kg REGN2810 IV infusion over 30 minutes each, every 21 days for 51 weeks
DL8: 10 mg/kg REGN3767 and 350 mg (fixed dose) REGN2810 IV infusion over 30 minutes every 21 days for 51 weeks.
DL9: 20 mg/kg REGN3767 and 350 mg REGN2810 IV infusion over 30 minutes each, every 21 days for 51 weeks
DL11: 40 mg/kg REGN3767 over 60 minutes and 350 mg REGN2810 IV infusion over 60 minutes every 21 days for 51 weeks
DL-1c: 0.3 mg/kg REGN3767 and 3.0 mg/kg REGN2810 IV infusion over 30 minutes each, every 21 days for 51 weeks (if necessary)
Combination therapy expansion cohorts: 1600 mg REGN3767 and 350 mg REGN2810 IV infusion over 30 minutes each, every 21 days for 51 weeks.

### Prohibited Medications

While participating in this study, a patient may not receive any standard or investigational agent for treatment of a tumor other than REGN3767 as monotherapy or in combination REGN2810, per the study's specified dosing regimens. Patients must not receive live vaccines during the study. Focal palliative treatment (e.g., radiation) may be allowed for local control of a tumor once a patient has completed 8 weeks of study treatment. Any other medication which is considered necessary for the patient's welfare, and which is not expected to interfere with the evaluation of the study drug, may be given.

Patients using immunosuppressive doses (>10 mg per day of prednisone or equivalent) of systemic corticosteroids other than for corticosteroid replacement are not eligible for the study. It is recommended that patients do not receive systemic corticosteroids such as hydrocortisone, prednisone, prednisolone (Solu-Medrol®) or dexamethasone (Decadron®) at any time throughout the study except in the case of a life-threatening emergency and/or to treat an irAE.

Similarly, it is recommended that patients do not receive other immunosuppressive medications (e.g., methotrexate) at any time throughout the study except in the case of a life-threatening emergency and/or to treat and irAE. Other immunosuppressive medications required to treat irAEs, infusion-related reactions, or life-threatening emergencies may be used. Adverse events requiring immunosuppressive medication may be treated with medications not specifically mentioned in the protocol.

### Permitted Medications

Physiologic replacement doses of systemic corticosteroids are permitted, even if >10 mg/day prednisone equivalents. A brief course of corticosteroids for prophylaxis (e.g., contrast dye allergy) or for treatment of non-autoimmune conditions (e.g., delayed-type hypersensitivity reaction caused by contact allergen) is permitted.

Gonadotropin-releasing hormone agonist therapy (e.g., for prostate cancer) may be continued and is not prohibited.

Treatments for bone metastases (bisphosphonates, denosumab) are not prohibited.

### Safety Procedures

An adverse event (AE) any untoward medical occurrence in a patient administered a study drug which may or may not have a causal relationship with the study drug. Therefore, an AE is any unfavorable and unintended sign (including abnormal laboratory finding), symptom, or disease which is temporally associated with the use of a study drug, whether or not considered related to the study drug. An AE also includes any worsening (i.e., any clinically significant change in frequency and/or intensity) of a preexisting condition that is temporally associated with the use of the study drug. Progression of underlying malignancy is not considered an AE if it is clearly consistent with the typical progression pattern of the underlying cancer (including time course, affected organs, etc.). Clinical symptoms of progression may be reported as AEs if the symptom cannot be determined as exclusively due to the progression of the underlying malignancy, or does not fit the expected pattern of progression for the disease under study. A serious AE (SAE) is any untoward medical occurrence that at any dose results in death, is life-threatening, requires hospitalization, results in persistent or significant disability, and/or is an important medical event.

Patients are monitored for vital signs, including temperature, resting blood pressure (seated), pulse, and respiratory rate. Patients are also monitored for anti-drug antibodies, monitored for changes in ECG from baseline, monitored for immune changes (e.g. changes in rheumatoid factor, thyroid stimulating hormone, and antinuclear antibody titer and pattern), monitored for coagulation changes, and B symptom changes.

REGN2810 (anti-PD-1), along with other checkpoint blockers (e.g., anti-CTLA-4) are associated with a unique set of toxicities termed immune-related adverse events (irAEs). Immune-related AEs are thought to be caused by unrestrained cellular immune responses directed at normal host tissues. An irAE can occur shortly after the first dose or several months after the last dose of treatment. Early detection and management reduces the risk of severe drug induced toxicity. Since LAG-3 is also a checkpoint molecule, anti-LAG-3 antibodies such as REGN3767 may also be associated with irAEs.

Early intervention may be needed in the management of irAEs, as the onset of symptoms of irAEs (e.g., pneumonitis) may be subtle.

When scheduled at the same visit as other procedures, vital signs should be measured prior to clinical laboratory assessments, pharmacokinetics, or exploratory sample collection.

### Efficacy Analysis

The following procedures are performed prior to the first dose of study drug for the purpose of determining study eligibility or characterizing the baseline population: height, serum pregnancy test, chest X ray (even if a CT is performed), brain CT or MRI, archival tumor sample collection, RF, ANA, and troponin, HPV testing (tumor). The chest X-ray is required to provide a baseline for any subsequent on-study chest X-rays (for example in the evaluation of potential pneumonitis). HPV status and sample collection for future HPV testing is for HNSCC patients only. Human immunodeficiency virus, hepatitis C, and hepatitis B are tested at screening. For patients with controlled HBV infection, periodic monitoring takes place while the patient is on study. For patients with controlled HIV or HCV infections, monitoring is performed per local standards.

Primary efficacy analysis includes best overall response determined by RECIST version 1.1 (Eisenhauer, 2009) for cohorts involving solid tumors and by Lugano criteria (Cheson, 2014) for DLBCL cohorts. Such results are summarized using descriptive statistics, along with 2-sided 95% confidence interval, by each expansion cohort.

The ORR is summarized by descriptive statistics, along with 95% confidence interval. Patients who are not evaluable for the BOR is considered as nonresponders.

For the given expansion cohort, if the number of responders is greater than or equal to the minimum number of responders specified in the Simon 2-stage design, the treatment is considered as effective and worthy of further investigation.

Within applicable expansion cohorts, the type I error is controlled by Simon 2-stage design. Adjustments to the significance level for the purposes of multiple testing are not applicable for the expansion cohorts. The statistical analyses of efficacy for these expansion cohorts is conducted and reported separately, i.e., efficacy results and clinical conclusions from each cohort does not affect the other cohorts, and vice versa.

The secondary analyses of efficacy include ORR as measured by iRECIST, DOR, rate of disease control and PFS. Those secondary efficacy endpoints are summarized descriptively by dose escalation and expansion cohorts.

### Efficacy Procedures

A CT or MRI for tumor assessment is performed at certain time points. Once the choice has been made to use CT scan or MRI, subsequent assessments should be made using the same modality whenever possible.

Tumor response assessments are performed according to RECIST version 1.1 criteria (Eisenhauer, 2009), Lugano criteria (lymphoma patients only) and irRC (Wolchok, 2009) (Table 3).

TABLE 3
Response According to Revised Response Evaluation
Criteria in Solid Tumors (Version 1.1)

| | | | | Best Overall |
|---|---|---|---|---|
| | | | | Response when |
| Target | Non-Target | New | Overall | Confirmation is |
| Lesions | Lesions | Lesions | Response | Required^{a)} |
| CR | CR | No | CR | ≥4 weeks |
| | | | | confirmation |
| CR | Non-CR/Non-PD | No | PR | ≥4 weeks |
| CR | Not evaluated | No | PR | confirmation |
| PR | Non-CR/Non-PD/ | No | PR | |
| | not evaluated | | | |
| SD | Non-CR/Non-PD/ | No | SD | Documented at |
| | not evaluated | | | least once ≥4 |
| | | | | weeks from |
| | | | | baseline |
| PD | Any | Yes or No | PD | no prior SD, |
| | | | | PR, or CR |
| Any | PD^{a} | Yes or No | PD | |
| Any | Any | Yes | PD | |

CR = complete response; PD = progressive disease; PR = partial response; SD = stable disease.
^{a)}In exceptional circumstances, unequivocal progression in non-target lesions may be accepted as PD.

Note: Patients with a global deterioration of health status requiring discontinuation of treatment without objective evidence of disease progression at that time should be reported as “symptomatic deterioration.” Every effort should be made to document the objective progression even after discontinuation of treatment.

Additional guidance for response assessment in CSCC patients: In some patients with unresectable locoregionally advanced basal cell carcinoma (BCC) or CSCC, it may not be possible to measure disease radiographically. For CSCC lesions that are not measurable radiographically, the approach from the phase II study of vismodegib in BCC is used (Sekulic, 2012). Response is defined at a decrease of 30% or more in externally visible or radiographic dimension (if applicable) or complete resolution of ulceration (if present at baseline). Residual scarring is to be included when measuring the externally visible dimension. Responses must be confirmed at least 4 weeks after the initial determination of response. Progressive disease is defined as an increase in 20% or more in the externally visible or radiographic dimension (if applicable), new ulceration, or new lesion (Sekulic. 2012). These criteria are applied to CSCC lesions that are not measurable radiographically in the current study. Central review of photographic images may be performed.

Response Assessment for Patients with Lymphoma

Disease response in patients with lymphoma is assessed using the Lugano criteria (Cheson 2014) (Table 4).

TABLE 4
Malignant Lymphoma Response Definitions per Lugano Criteria

| | FDG-PET-CT | |
|---|---|---|
| CMR/CR | based Response | CT-based response |
| | Complete Metabolic | Complete Radiologic |
| | Response (CMR) | response (CR) |
| | | (ALL of the following) |
| Target | Score 1, 2, or 3 with | Nodal Disease: ≤1.5 cm |
| Nodal/ | or without a residual | in LDi Extranodal |
| Extranodal | mass on 5-PS | Disease: Absent |
| Non-Target | | |
| Spleen | | Regress to normal |
| New Lesions | | None |
| Bone Marrow | | Normal by morphology; |
| | | if indeterminate, IHC |
| | | negative |

| | FDG-PET-CT | |
|---|---|---|
| PMR/PR | based Response | CT-based response |
| | Partial Metabolic | Partial Remission (PR) |
| | Response (PMR) | (ALL of the following) |
| Target | Score 4, 5 with reduced | ≥50% decrease from |
| Nodal/ | uptake compared with | baseline in SPD of |
| Extranodal | baseline and residual | all Target Lesions |
| Non-Target | mass(es) of any size | No Increase |
| Spleen | Interim: suggest | ≥50% decrease from |
| | responding to disease | baseline in enlarged |
| | EoT: Indicate residual | portion of spleen |
| | disease | (value over 13 cm) |
| New Lesions | | None |
| Bone Marrow | Residual uptake higher | Not applicable |
| | than uptake in normal | |
| | marrow but reduced | |
| | compared with baseline | |
| | Persistent focal changes | |
| | in the marrow with nodal | |
| | response, | |
| | Further evaluation with | |
| | MRI or biopsy, or an | |
| | interval scan | |

| | FDG-PET-CT | |
|---|---|---|
| NMR/SD | based Response | CT-based response |
| | No Metabolic | Stable disease (SD) |
| | Response (NMR) | |
| Target | Score 4, 5 with no | <50% decrease from |
| Nodal/ | significant change in | baseline in SPD of |
| Extranodal | FDG-PET-CT uptake | all Target lesions |
| | from baseline, at | No criteria for PD |
| | interim or EoT. | are met |
| Non-Target | | No progression |
| Spleen | | No progression |
| New Lesions | | None |
| Bone Marrow | No change from baseline | Not applicable |

| | FDG-PET-CT | |
|---|---|---|
| PMD/PD | based Response | CT-based response |
| | Progressive Metabolic | Progressive disease |
| | Disease (PMD) | One of the following |
| Target | Score 4, 5 with no | PPD Progression: |
| Nodal/ | significant change in | An individual node/lesion |
| Extranodal | FDG-PET-CT uptake | must be abnormal with: |
| | from baseline, at | LDi >1.5 cm AND |
| | interim or EoT. | Increase by ≥50% from |
| | | PPD nadir AND |
| | | An increase in LDi or |
| | | SDi from nadir |
| | | >0.5 cm for lesions <2 cm |
| | | > 1.0 cm for lesions >2 cm |
| Non-Target | | Unequivocal Progression |
| Spleen | | No progression |
| New Lesions | | Progression of existing |
| | | splenomegaly |
| | | New or Recurrent |
| | | Splenomegaly |
| Bone Marrow | No change from baseline | New/recurrent involvement |

Abbreviations
5PS: 5-point scale
CT: Computed tomography
FDG: fluorodeoxyglucose
IHC: immunohistochemistry
LDi: Longest transverse Diameter of a lesion
MRI: Magnetic Resonance Imaging
PET: positron emission tomography
PPD: Cross product of the LDi and perpendicular diameter
SDI: shortest axis perpendicular to the LDi
SPD: sum of the product of the perpendicular diameters for multiple lesions
†PET 5PS:
1: no uptake above background;
2: uptake ≤ mediastinum
3: uptake > mediastinum but ≤ liver
4: uptake moderately > liver
5: uptake markedly higher than liver and/or new lesions;
X: new areas of uptake unlikely to be related to lymphoma

A CT or MRI scan of the neck, chest, abdomen, pelvis, liver, and spleen, as well as any other known sites of disease, is performed at certain time points and at any time when disease progression is suspected. Scans include description of nodal locations, and the 6 largest dominant nodes or nodal disease masses should be chosen as indicator lesions and measured in perpendicular dimensions. Tumor lesion assessments include 2-dimensional diameters of all lymph nodes, spleen, and liver enlargement. All measurable and evaluable lesions should be assessed and documented.

PET scans are performed at specified time points. FDG-PET-CT is allowed for disease evaluation if FDG-PET-CT slice thickness is 5 mm, and reliable measurements of the lesions can be measured and recorded.

Collection of a bone marrow sample is optional for patients with lymphoma and is performed at specified time points.

### Procedures and Assessments

The safety and tolerability of REGN3767 alone or in combination with REGN2810 is monitored by clinical assessment of AEs and by repeated measurements of clinical evaluation including vital signs (temperature, blood pressure, pulse, and respiration), physical examinations (complete and limited), 12-lead electrocardiograms (ECGs), and laboratory assessment including standard hematology, chemistry, and urinalysis.

Blood samples for the determination of functional REGN3767 and functional REGN2810 in serum and ADA (anti REGN3767 or anti REGN2810) samples are collected.

Serum and plasma samples are collected for analysis of additional biomarkers. Clinical activity; or underlying disease is investigated in serum, plasma, peripheral blood mononuclear cells (PBMCs), and tumor tissue.

Anti-tumor activity is assessed by Positron Emission Tomography (PET) computed tomography (CT), CT and magnetic resonance imaging (MRI).

A genomic DNA sample is collected from patients who have consented to the optional pharmacogenomics sub-study.

### Study Endpoints

In the Dose Escalation Phase, the primary endpoint is safety, including Rate of DLTs, adverse events (AEs; including immune-related), serious adverse events (SAEs), deaths, and laboratory abnormalities (grade 3 or higher per Common Terminology Criteria for Adverse Events [CTCAE]), and pharmacokinetics. In the Dose Expansion Phase, the primary endpoint is objective response rate (ORR) based on RECIST 1.1 (solid tumors) and Lugano criteria (lymphoma). The secondary endpoints include: objective response rate on RECIST 1.1 (solid tumors) and Lugano criteria (lymphoma) (for the escalation phase); best overall response (BOR), duration of response (DOR), disease control rate, and progression free survival (PFS) based on RECIST, irRC, and Lugano criteria; AEs; including immune-related, SAEs, deaths, and laboratory abnormalities (grade 3 or higher per CTCAE); and pharmacokinetics and ADA.

### Results

In this study, initial safety, pharmacokinetics (PK), and efficacy of the dose escalation study of an exemplary anti-LAG-3 antibody, REGN3767, alone (mono) or in combination with an exemplary anti-PD-1 antibody, REGN2810; (cemiplimab-rwlc), was determined in patients with advanced malignancies.

Patients who had progressed on prior therapy(ies) and/or for whom no therapy with clinical benefit available were enrolled; most patients enrolled in the dose escalation cohorts had received no prior anti-PD-1/PD-L1 treatment. Patients received REGN3767 at doses of 1, 3, 10, or 20 mg/kg every 3 weeks (Q3W) and REGN2810 at doses of 3 mg/kg or 350 mg Q3W IV for 51 weeks. Crossover from monotherapy REGN3767 to combination with REGN2810 (cemiplimab) was allowed at progression. Pharmacokinetics of REGN3767 were evaluated. Tumor measurements were performed every 6 weeks for the first 24 weeks and subsequently every 9 weeks.

Patient Characteristics and Disposition: The monotherapy cohort included 27 patients with a median age of 68 years (range from 22-83 years) and having an ECOG PS (ECOG Performance Status, a measure of the ability of patient to tolerate chemotherapy) of 0 in 4 patients and 1 in 23 patients. The combination therapy cohort included 42 patients with a median age of 60 years (range from 30-83 years) and having an ECOG PS of 0 in 15 patients and 1 in 27 patients (Tables 5 and 6).

TABLE 5
Patient Characteristics

| | REGN3767 | | REGN3767+ | | Mono to | |
|---|---|---|---|---|---|---|
| | monotherapy | | cemiplimab | | combo | |
| | (N = 27) | | (N = 42) | | (N = 13) | |
| Median age, years (range) | 68 | (22-83) | 60 | (30-83) | 64 | (22-83) |
| Female, n (%) | 14 | (51.9) | 24 | (57.1) | 7 | (53.8) |
| ECOG PS 0; 1, n (%) | 4 | (14.8); | 15 | (35.7); | 3 | (23.1); |
| | 23 | (85.2) | 27 | (64.3) | 10 | (76.9) |

Most common primary site of cancer n (%)

| Colon | 2 | (7.4) | 10 | (23.8) | 2 | (15.4) |
|---|---|---|---|---|---|---|
| Lung^{†} | 2 | (7.4) | 4 | (9.5) | 1 | (7.7) |
| Biliary tract | 1 | (3.7) | 3 | (7.1) | 0 | |
| Ovary | 2 | (7.4) | 2 | (4.8) | 1 | (7.7) |

| | | | | | |
|---|---|---|---|---|---|
| Pancreas | 1 | (3.7) | 3 | (7.1) | 0 |

Prior lines of systemic therapy, n (%)

| Any | 25 | (92.6) | 42 | (100) | 12 | (92.3) |
|---|---|---|---|---|---|---|
| 1-2 | 9 | (33.3) | 16 | (38.1) | 7 | (53.8) |
| ≥3 | 16 | (59.3) | 26 | (61.9) | 5 | (38.5) |
| Prior anti-PD-1/PD-L1 | 0 | | 3 | (7.2) | 0 | |
| Prior radiotherapy | 15 | (55.6) | 27 | (64.3) | 9 | (69.2) |
| ≥3 | 16 | (59.3) | 26 | (61.9) | 5 | (38.5) |
| Prior anti-PD-1/PD-L1 | 0 | | 3 | (7.2) | 0 | |
| Prior radiotherapy | 15 | (55.6) | 27 | (64.3) | 9 | (69.2) |

TABLE 6
Patient Disposition

| | REGN3767 | | REGN3767+ | | Mono to | |
|---|---|---|---|---|---|---|
| | monotherapy | | cemiplimab | | combo | |
| n (%) | (N = 27) | | (N = 42) | | (N = 13) | |
| Completed treatment | 1 | (3.7) | 0 | | 0 | |
| Discontinued | 26 | (96.3) | 41 | (97.6) | 13 | (100) |
| treatment | | | | | | |

Primary reason for discontinuing treatment

| | | | | | | |
|---|---|---|---|---|---|---|
| Disease progression | 16 | (59.3) | 36 | (85.7) | 8 | (61.5)^{†} |

| Death^{‡} | 4 | (14.8) | 3 | (7.1) | 0 |
|---|---|---|---|---|---|
| Other | 3 | (11.1) | 0 | | 0 |
| Withdrawal of consent | 2 | (7.4) | 1 | (2.4) | 0 |

| | | | | |
|---|---|---|---|---|
| Investigator's | 1 | (3.7) | 0 | 0 |
| decision | | | | |

| | | | | | |
|---|---|---|---|---|---|
| Patient decision | 0 | 1 | (2.4) | 2 | (15.4) |

| | | | | |
|---|---|---|---|---|
| Adverse event | 0 | 0 | 3 | (23.1) |

Table 7 shows patient exposure to REGN3767.

TABLE 7
Exposure to REGN3767

| | REGN3767 | | REGN3767+ | | Mono to | |
|---|---|---|---|---|---|---|
| | monotherapy | | cemiplimab | | combo | |
| | (N = 27) | | (N = 42) | | (N = 13) | |
| Duration of exposure, | 12.0 | (2-51) | 9.1 | (3-80) | 16.7 | (6-66) |
| weeks, median (range) | | | | | | |
| Number of doses of | 4 | (1-17) | 3 | (1-23) | 6 | (2-20) |
| study drug, median | | | | | | |
| (range) | | | | | | |

Safety: Treatment-emergent adverse events (TEAEs) recorded in patient treated with monotherapy, combo therapy and patients who crossed from monotherapy to combination therapy are shown in Tables 8-10.

TABLE 8
TEAEs in Patients treated with REGN3767 Monotherapy

| | REGN3767 monotherapy | | |
|---|---|---|---|
| TEAEs regardless of | (N = 27) | | |
| attribution, n (%) | | Any grade | Grade ≥3 |

| | | | | |
|---|---|---|---|---|
| Any | 23 | (85.2) | 11 | (40.7) |
| Serious | 6 | (22.2) | 5 | (18.5) |
| Immune-related | 2 | (7.4) | 1 | (3.7) |

| | | |
|---|---|---|
| Led to discontinuation | 0 | 0 |

| | | | | |
|---|---|---|---|---|
| With an outcome of death | 1 | (3.7) | 1 | (3.7) |

Most common (occurred in ≥15% of patients) by any grade

| Abdominal pain | 5 | (18.5) | 0 | |
|---|---|---|---|---|
| Decreased appetite | 5 | (18.5) | 0 | |
| Diarrhea | 5 | (18.5) | 0 | |
| Fatigue | 5 | (18.5) | 0 | |
| Nausea | 5 | (18.5) | 1 | (3.7) |
| Vomiting | 5 | (18.5) | 1 | (3.7) |

TABLE 9
TEAEs in Patients treated with REGN3767 + Cemiplimab

| | REGN3767 + cemiplimab | | |
|---|---|---|---|
| TEAEs regardless of | (N = 42) | | |
| attribution, n (%) | | Any grade | Grade ≥3 |

| | | | | |
|---|---|---|---|---|
| Any | 38 | (90.5) | 19 | (45.2) |
| Serious | 9 | (21.4) | 9 | (21.4) |
| Immune-related | 14 | (33.3) | 3 | (7.1) |

| | | |
|---|---|---|
| Led to discontinuation | 0 | 0 |

| | | | | |
|---|---|---|---|---|
| With an outcome of death | 1 | (2.4) | 1 | (2.4) |

Most common (occurred in ≥15% of patients) by any grade

| Fatigue | 16 | (38.1) | 1 | (2.4) |
|---|---|---|---|---|
| Nausea | 10 | (23.8) | 0 | |
| Vomiting | 9 | (21.4) | 1 | (2.4) |
| Anemia | 7 | (16.7) | 3 | (7.1) |
| Chills | 7 | (16.7) | 0 | |
| Decreased appetite | 7 | (16.7) | 2 | (4.8) |
| Diarrhea | 7 | (16.7) | 0 | |
| Headache | 7 | (16.7) | 0 | |
| Hypothyroidism | 7 | (16.7) | 1 | (2.4) |

TABLE 10
TEAEs in Patients who crossed from
Monotherapy to Combination therapy

| | Mono to combo | | |
|---|---|---|---|
| TEAEs regardless of | (N = 13) | | |
| attribution, n (%) | | Any grade | Grade ≥3 |

| | | | | |
|---|---|---|---|---|
| Any | 11 | (84.6) | 6 | (46.2) |
| Serious | 3 | (23.1) | 3 | (23.1) |
| Immune-related | 9 | (69.2) | 3 | (23.1) |
| Led to discontinuation | 3 | (23.1) | 2 | (15.4) |

| | | |
|---|---|---|
| With an outcome of death | 0 | 0 |

Most common (occurred in ≥15% of patients) by any grade

| Fatigue | 5 | (38.5) | 0 | |
|---|---|---|---|---|
| Maculo-papular rash | 5 | (38.5) | 2 | (15.4) |
| Nausea | 5 | (38.5) | 1 | (7.7) |
| Diarrhea | 4 | (30.8) | 0 | |
| Decreased appetite | 3 | (23.1) | 1 | (7.7) |
| Peripheral edema | 3 | (23.1) | 0 | |
| Hypokalemia | 2 | (15.4) | 2 | (15.4) |

| | | | |
|---|---|---|---|
| Other^{†} | 2 | (15.4) | 0 |

There were no DLTs seen in the patients treated with REGN3767 monotherapy or in patients that crossed from monotherapy to combination therapy.

Pharmacokinetics: REGN3767 concentrations increased in a dose-dependent manner and were unaffected by combination with REGN2810. REGN3767 exposure is similar at 1600 mg Q3W compared to 20 mg/kg Q3W.

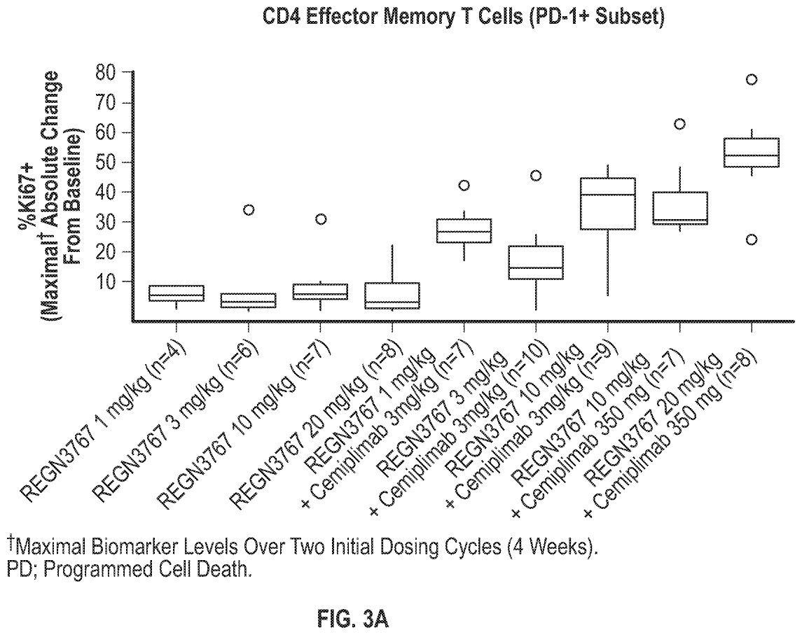

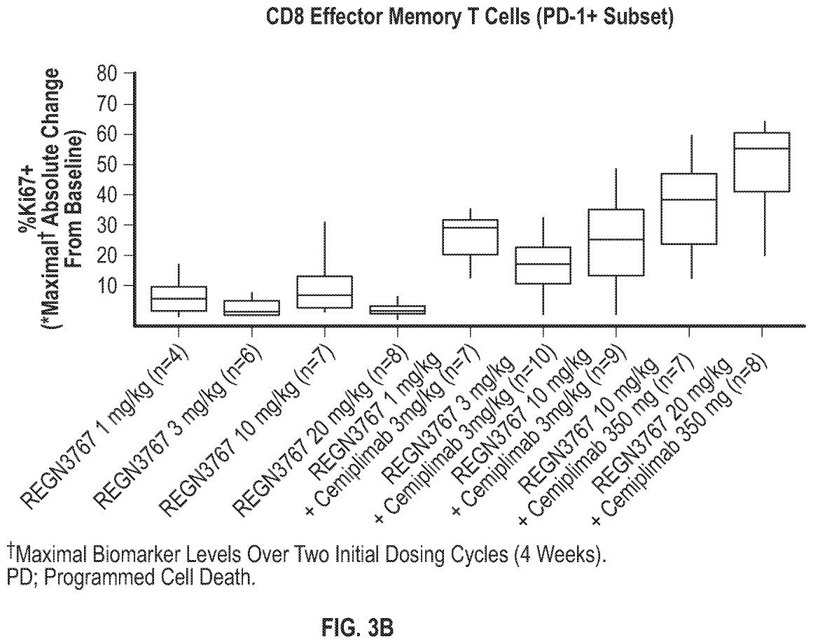

Efficacy: A trend of higher PD-1+ effector memory T-cell proliferation was observed with increasing REGN3767 dose in the cemiplimab combination cohorts (FIG. 3). Table 11 shows preliminary assessment of response rates in patients in the monotherapy and combination cohorts.

TABLE 11
Investigator assessed Preliminary Response rate by RECIST 1.1

| | | |
|---|---|---|
| REGN3767 | REGN3767+ | Mono to |
| monotherapy | cemiplimab | combo |
| (N = 25)^{†} | (N = 42) | (N = 12)^{†} |

Best overall
response, n (%)

| Partial response | 0 | | 2 | (4.8)^{‡} | 2 | (16.7)^{§} |
|---|---|---|---|---|---|---|
| Stable disease^{#} | 12 | (48.0) | 11 | (26.2) | 6 | (50.0) |
| Progressive disease | 7 | (28.0) | 24 | (57.1) | 3 | (25.0) |
| Unknown^{¶} | 6 | (24.0) | 5 | (11.9) | 1 | (8.3) |

| Range of DOR, months | NA | 3.5 to 13.6+ | | 12.5+ to 17.3+ | |
|---|---|---|---|---|---|
| Patients with ongoing | NA | 1 | (50.0) | 2 | (100) |
| response, n (%)^{††} | | | | | |

Two patients with small cell lung cancer had a partial response, with one of the patients showing an ongoing prolonged response (>12 months). A patient with cholangiocarcinoma showed tumor shrinkage and stable disease after several months after progression on cemiplimab monotherapy. A patient with endometrial cancer and another with cutaneous squamous cell carcinoma both had a partial response. Patients with partial responses showed durable responses (>1 year).

Conclusions: The safety profile of REGN3767 in combination with REGN2810 was generally tolerable. There were no new safety signals with REGN3767 monotherapy or REGN3767+ cemiplimab compared with those previously reported for cemiplimab. REGN3767 concentrations increased in a dose-dependent manner and were unaffected by co-administration with cemiplimab. There was no pharmacodynamic effect on peripheral T-cells observed for REGN3767 monotherapy. Preliminary data suggest a dose-dependent relationship between REGN3767+ cemiplimab dosing and generation of PD-1 expressing memory T cell subsets. Early efficacy signals were detected despite the very difficult-to-treat patient population. REGN3767 20 mg/kg or 1600 mg fixed dose equivalent Q3W as monotherapy and in combination with cemiplimab were selected for further evaluation.

### Results in the Dose Expansion Cohorts

Results to date show promising efficacy in melanoma patients for the R3767 and cemiplimab combination. The overall response for the PD-(L)1-naïve melanoma patients was 66.7%, with partial responses seen in 6 of 9 patients. In the PD-(L)1-experienced patient population with melanoma, 2PR were observed of 15 patients (ORR 13.3%). Numerous partial responses were also observed in both PD-(L)1-naïve and PD-(L)1-experienced patients with squamous cell carcinoma of head and neck (HNSCC), renal cell carcinoma, NSCLC and DLBCL, as shown in Tables 12 and 13, below.

TABLE 12
Overall Tumor Response Rate by Investigator Assessment per RECIST 1.1 in PD-(L)1-Naïve Patients

| | Exp1 | | Exp3 | | Exp6 | | Exp11 | |
|---|---|---|---|---|---|---|---|---|
| | NSCLC | | ccRCC | | Melanoma | | HNSCC | |
| | (N = 15) | | (N = 15) | | (N = 9) | | (N = 15) | |
| Best Overall Response (BOR) | | | | | | | | |
| Complete Response (CR) | 0 | | 0 | | 0 | | 0 | |
| Partial Response (PR) | 4 | (26.7%) | 3 | (20.0%) | 6 | (66.7%) | 5 | (33.3%) |
| Stable Disease (SD) | 3 | (20.0%) | 6 | (40.0%) | 0 | | 2 | (13.3%) |
| Progressive Disease (PD) | 8 | (53.3%) | 5 | (33.3%) | 2 | (22.2%) | 5 | (33.3%) |
| Not evaluated (NE) | 0 | | 1 | (6.7%) | 1 | (11.1%) | 3 | (20.0%) |
| Response | | | | | | | | |
| Objective Response Rate | 4 | (26.7%) | 3 | (20.0%) | 6 | (66.7%) | 5 | (33.3%) |
| (ORR) (CR + PR) | | | | | | | | |
| 95% CI for ORR | (7.8%, 55.1%) | | (4.3%, 48.1%) | | (29.9%, 92.5%) | | (11.8%, 61.6%) | |
| Disease Control Rate (DCR) | 7 | (46.7%) | 9 | (60.0%) | 6 | (66.7%) | 7 | (46.7%) |
| (CR + PR + SD) | | | | | | | | |

| | | | | |
|---|---|---|---|---|
| 95% CI for DCR | (21.3%, 73.4%) | (32.3%, 83.7%) | (29.9%, 92.5%) | (21.3%, 73.4%) |

TABLE 13
Overall Tumor Response Rate by Investigator Assessment
per RECIST 1.1 in PD-(L)1-Experienced Patients

| | Exp2 | | Exp4 | | Exp7 | | Exp12 | |
|---|---|---|---|---|---|---|---|---|
| | NSCLC | | ccRCC | | Melanoma | | HNSCC | |
| | (N = 15) | | (N = 12) | | (N = 15) | | (N = 15) | |
| Best Overall Response (BOR) | | | | | | | | |
| Complete Response (CR) | 0 | | 0 | | 0 | | 0 | |
| Partial Response (PR) | 1 | (6.7%) | 1 | (8.3%) | 2 | (13.3%) | 2 | (13.3%) |
| Stable Disease (SD) | 6 | (40.0%) | 8 | (66.7%) | 4 | (26.7%) | 8 | (53.3%) |
| Progressive Disease (PD) | 6 | (40.0%) | 3 | (25.0%) | 8 | (53.3%) | 4 | (26.7%) |
| Not evaluated (NE) | 2 | (13.3%) | 0 | | 1 | (6.7%) | 1 | (6.7%) |
| Response | | | | | | | | |
| Objective Response Rate | 1 | (6.7%) | 1 | (8.3%) | 2 | (13.3%) | 2 | (13.3%) |
| (ORR) (CR + PR) | | | | | | | | |
| 95% CI for ORR | (0.2%, 31.9%) | | (0.2%, 38.5%) | | (1.7%, 40.5%) | | (1.7%, 40.5%) | |
| Disease Control Rate (DCR) | 7 | (46.7%) | 9 | (75%) | 6 | (40.0%) | 10 | (66.7%) |
| (CR + PR + SD) | | | | | | | | |

In general, response was observed in patients with CSCC and DLBCL (unconfirmed partial responses) at the time of filing.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims.

We claim:

1. A method of treating cancer or inhibiting the growth of a tumor comprising administering to a subject in need thereof a therapeutically effective amount each of (a) an antibody or antigen-binding fragment thereof that specifically binds programmed death 1 (PD-1); and (b) an antibody or antigen-binding fragment thereof that specifically binds lymphocyte activation gene-3 (LAG-3).

2. The method of claim 1, wherein the anti-PD-1 antibody comprises 50 to 1500 mg.

3. The method of claim 1 or 2, wherein the anti-PD-1 antibody comprises 350 mg.

4. The method of any one of claims 1 to 3, wherein the anti-LAG-3 antibody comprises between 0.1 mg/kg and 50 mg/kg of the subject's body weight.

5. The method of any one of claims 1 to 3, wherein the therapeutically effective amount of the anti-LAG-3 antibody comprises 50 to 8000 mg.

6. The method of any one of claims 1 to 5, wherein the anti-LAG-3 antibody is administered prior to, concurrent with or after the anti-PD-1 antibody.

7. The method of claim 6, wherein the anti-LAG-3 antibody is administered prior to the anti-PD-1 antibody.

8. The method of claim 6, wherein the anti-LAG-3 antibody is administered the same day as the anti-PD-1 antibody.

9. The method of any one of claims 1-8, wherein one or more doses of the anti-LAG-3 antibody are administered in combination with one or more doses of the anti-PD-1 antibody.

10. The method of claim 9, wherein each dose of the anti-PD-1 antibody comprises 0.3, 1, 3, or 10 mg/kg of the subject's body weight.

11. The method of claim 9, wherein each dose of the anti-PD-1 antibody comprises 350 mg.

12. The method of any one of claims 9 to 11, wherein each dose of the anti-LAG-3 antibody comprises between 0.1 mg/kg and 50 mg/kg of the subject's body weight.

13. The method of claim any one of claims 9 to 11, wherein each dose of the anti-LAG-3 antibody comprises between 50 and 8000 mg.

14. The method of claim 12, wherein each dose of the anti-PD-1 antibody comprises 1, 3 or 10 mg/kg and each dose of the anti-LAG-3 antibody comprises 1, 3, 10, 20, 30, or 40 mg/kg of the subject's body weight.

15. The method of claim 13, wherein each dose of the anti-PD-1 antibody comprises 200 mg, 250 mg or 350 mg and each dose of the anti-LAG-3 antibody comprises 800 mg, 1000 mg, 1400 mg, or 1600 mg.

16. The method of any one of claims 9 to 15, wherein each dose of the anti-PD-1 antibody is administered 0.5 weeks to 12 weeks after the immediately preceding dose.

17. The method of any one of claims 9 to 16, wherein each dose of the anti-LAG-3 antibody is administered 0.5 weeks to 12 weeks after the immediately preceding dose.

18. The method of any one of claims 9 to 17, wherein each dose of the anti-PD-1 antibody is administered once in three weeks or once in six weeks.

19. The method of any one of claims 9 to 18, wherein each dose of the anti-LAG-3 antibody is administered once in three weeks or once in six weeks.

20. The method of any one of claims 1 to 19, wherein the antibodies are administered intravenously, subcutaneously, or intraperitoneally.

21. The method of any one of claims 1 to 20, wherein the cancer is selected from the group consisting of astrocytoma, bladder cancer, blood cancer, blood cancer, bone cancer, brain cancer, breast cancer, cervical cancer, clear cell renal cell carcinoma, colorectal cancer, microsatellite-intermediate colorectal cancer, cutaneous squamous cell carcinoma, diffuse large B-cell lymphoma, endometrial cancer, esophageal cancer, fibrosarcoma, gastric cancer, glioblastoma, glioblastoma multiforme, head and neck squamous cell carcinoma, hepatic cell carcinoma, leukemia, liver cancer, leiomyosarcoma, lung cancer, lymphoma, melanoma, mesothelioma, myeloma, nasopharyngeal cancer, non-small cell lung cancer, osteosarcoma, ovarian cancer, pancreatic cancer, primary and/or recurrent cancer, prostate cancer, renal cell carcinoma, rhabdomyosarcoma, small cell lung cancer, squamous cell cancer, synovial sarcoma, thyroid cancer, triple negative breast cancer, uterine cancer, and Wilms' tumor.

22. The method of any one of claims 1 to 21, wherein the subject has received prior anti-cancer therapy comprising one or more of a PD-1 inhibitor, a PD-L1 inhibitor, surgery, radiation therapy or chemotherapy.

23. The method of any one of claims 1 to 22, wherein the subject is resistant or inadequately responsive to, or relapsed after prior therapy.

24. The method of any one of claims 1 to 21, wherein the subject has not received prior anti-cancer therapy.

25. The method of claim 22, wherein the prior anti-cancer therapy comprises a PD-1 inhibitor or a PD-L1 inhibitor.

26. The method of any one of claims 1 to 25, wherein the treatment produces a therapeutic effect selected from the group consisting of delay in tumor growth, reduction in tumor cell number, tumor regression, increase in survival, partial response, and complete response.

27. The method of claim 26, wherein tumor growth is delayed by at least 10 days as compared to an untreated subject.

28. The method of claim 26, wherein the tumor growth is inhibited by at least 50% as compared to an untreated subject.

29. The method of claim 26, wherein the tumor growth is inhibited by at least 20% as compared to a subject administered with either antibody as monotherapy.

30. The method of any one of claims 1 to 29 further comprising administering to the subject a third therapeutic agent or therapy, wherein the third therapeutic agent or therapy is selected from the group consisting of radiation, surgery, a chemotherapeutic agent, a cancer vaccine, a PD-L1 inhibitor, a CTLA-4 inhibitor, a TIM3 inhibitor, a BTLA inhibitor, a TIGIT inhibitor, a CD47 inhibitor, a CD28 agonist, a CD38 inhibitor, an indoleamine-2,3-dioxygenase (IDO) inhibitor, a vascular endothelial growth factor (VEGF) antagonist, an angiopoietin-2 (Ang2) inhibitor, a transforming growth factor beta (TGFβ) inhibitor, an epidermal growth factor receptor (EGFR) inhibitor, an antibody to a tumor-specific antigen, Bacillus Calmette-Guerin vaccine, granulocyte-macrophage colony-stimulating factor, an oncolytic virus, a cytotoxin, an interleukin 6 receptor (IL-6R) inhibitor, an interleukin 4 receptor (IL-4R) inhibitor, an IL-10 inhibitor, IL-2, IL-7, IL-21, IL-12, IL-15, an antibody-drug conjugate, a GITR agonist, a 4-1 BB agonist, CD20×CD3 bispecific antibody, MUC16×CD3 bispecific antibody, and an anti-inflammatory drug.

31. The method of any one of claims 1 to 30, wherein the anti-PD-1 antibody or antigen-binding fragment thereof comprises the heavy chain complementarity determining regions (HCDR1, HCDR2 and HCDR3) of a heavy chain variable region (HCVR) and three light chain complementarity determining regions (LCDR1, LCDR2 and LCDR3) of a light chain variable region (LCVR), wherein HCDR1 comprises the amino acid sequence of SEQ ID NO: 3; HCDR2 comprises the amino acid sequence of SEQ ID NO: 4; HCDR3 comprises the amino acid sequence of SEQ ID NO: 5; LCDR1 comprises the amino acid sequence of SEQ ID NO: 6; LCDR2 comprises the amino acid sequence of SEQ ID NO: 7; and LCDR3 comprises the amino acid sequence of SEQ ID NO: 8.

32. The method of claim 31, wherein the HCVR comprises the amino acid sequence of SEQ ID NO: 1 and the LCVR comprises the amino acid sequence of SEQ ID NO: 2.

33. The method of any one of claims 1 to 32, wherein the anti-PD-1 antibody comprises a heavy chain comprising the amino acid sequence of SEQ ID NO: 9 and a light chain comprising the amino acid sequence of SEQ ID NO: 10.

34. The method of any one of claims 1 to 33, wherein the anti-LAG-3 antibody or antigen-binding fragment thereof comprises the heavy chain CDRs (HCDR1, HCDR2 and HCDR3) of a HCVR and three light chain CDRs (LCDR1, LCDR2 and LCDR3) of a LCVR, wherein HCDR1 comprises the amino acid sequence of SEQ ID NO: 13; HCDR2 comprises the amino acid sequence of SEQ ID NO: 14; HCDR3 comprises the amino acid sequence of SEQ ID NO: 15; LCDR1 comprises the amino acid sequence of SEQ ID NO: 16; LCDR2 comprises the amino acid sequence of SEQ ID NO: 17; and LCDR3 comprises the amino acid sequence of SEQ ID NO: 18.

35. The method of claim 34, wherein the HCVR comprises the amino acid sequence of SEQ ID NO: 11 and the LCVR comprises the amino acid sequence of SEQ ID NO: 12.

36. The method of any one of claims 1 to 35, wherein the anti-LAG-3 antibody comprises a heavy chain comprising the amino acid sequence of SEQ ID NO: 19 and a light chain comprising the amino acid sequence of SEQ ID NO: 20.

37. The method of any one of claims 1 to 36, wherein the inhibition is more efficacious than administration of either antibody as a monotherapy.

38. A method of treating cancer or inhibiting the growth of a tumor comprising:
(1) selecting a patient with a tumor, wherein the selected patient has received prior treatment comprising a PD-1 inhibitor or PD-L1 inhibitor; and
(2) administering to the patient (a) 350 mg anti-PD-1 antibody comprising the HCVR/LCVR amino acid sequence pair of SEQ ID NOs: 1/2; and (b) 1, 3, 10, 20, or 40 mg/kg or 1600 mg anti-LAG-3 antibody comprising the HCVR/LCVR amino acid sequence pair of SEQ ID NOs: 11/12.

39. The method of claim 38, wherein the administering of step (2) occurs once every 3 weeks, or once every 6 weeks.

40. The method of claim 38, wherein the patient is further selected as having one or more of the following criteria:
(i) ineligible for platinum based therapy, or tumor progression or recurrence within 6 months of last dose of platinum therapy;
(ii) confirmed diagnosis of malignancy;
(iii) demonstrated progression of a tumor for which there is no available therapy likely to convey clinical benefit;
(iv) disease progression/recurrence after one platinum-containing regimen;
(v) anti-PD-1/PD-L1 experienced stage IIIB, IIIC, or IV NSCLC with no more than 2 prior therapies for metastatic disease;
(vi) anti-PD-1/PD-L1 experienced advanced or metastatic ccRCC with a clear cell component who had received no more than 2 previous regimens of anti-angiogenic therapy;
(vii) anti-PD-1/PD-L1 experienced advanced or metastatic non-uveal melanoma who have received no more than 2 previous regimens for metastatic disease;
(xiii) anti-PD-1/PD-L1 experienced relapsed/refractory DLBCL who have either progressed after or are not candidates for autologous stem cell transplant;
(ix) anti-PD-1/PD-L1 experienced recurrent and/or metastatic HNSCC (irrespective of HPV status) with no curative options;
(x) anti-PD-1/PD-L1 experienced locally advanced or metastatic CSCC not be appropriate for surgery; and
(xi) the patient has 1% LAG-3 expression in tumor tissue, wherein the tumor tissue comprises tumor cells and tumor-infiltrating immune cells.

41. A method of treating cancer or inhibiting the growth of a tumor comprising:
(1) selecting a patient with a tumor, wherein the selected patient has not received prior treatment with a PD-1 inhibitor or PD-L1 inhibitor; and
(2) administering to the patient (a) 350 mg anti-PD-1 antibody comprising the HCVR/LCVR amino acid sequence pair of SEQ ID NOs: 1/2; and (b) 1, 3, 10, 20, or 40 mg/kg or 1600 mg anti-LAG-3 antibody comprising the HCVR/LCVR amino acid sequence pair of SEQ ID NOs: 11/12.

42. The method of claim 41, wherein the administering of step (2) occurs once every 3 weeks, or once every 6 weeks.

43. The method of claim 41, wherein the patient is further selected as having one or more of the following criteria:
(i) ineligible for platinum based therapy, or who have had tumor progression or recurrence within 6 months of last dose of platinum therapy;
(ii) confirmed diagnosis of malignancy;
(iii) demonstrated progression of a tumor for which there is no available therapy likely to convey clinical benefit;
(iv) anti-PD-1/PD-L1 naïve stage IIIB, IIIC, or IV NSCLC either without prior therapy for metastatic disease;
(v) disease progression/recurrence after one platinum-containing regimen;
(vi) anti-PD-1/PD-L1 naïve advanced or metastatic ccRCC with a clear cell component who had received no more than 2 previous regimens of anti-angiogenic therapy;
(vii) anti-PD-1/PD-L1 naïve metastatic TNBC (estrogen, progesterone, and human epidermal growth factor receptor 2 negative) who have received 5 or fewer prior lines of therapy;
(viii) anti-PD-1/PD-L1 naïve advanced or metastatic non-uveal melanoma who have received no more than 2 previous regimens for metastatic disease;
(ix) anti-PD-1/PD-L1 naïve relapsed/refractory DLBCL who have either progressed after or are not candidates for autologous stem cell transplant;
(x) anti-PD-1/PD-L1 naïve recurrent and/or metastatic HNSCC (irrespective of HPV status) with no curative options;
(xi) anti-PD-1/PD-L1 naïve locally advanced or metastatic CSCC not be appropriate for surgery; and
(xii) the patient has 1% LAG-3 expression in tumor tissue, wherein the tumor tissue comprises tumor cells and tumor-infiltrating immune cells.

44. A method of treating cancer or inhibiting the growth of a tumor comprising:
(1) selecting a patient with a tumor; and
(2) administering to the patient (a) 1, 3, 10, 20, or 40 mg/kg anti-LAG-3 antibody comprising the HCVR/LCVR amino acid sequence pair of SEQ ID NOs: 11/12 as a monotherapy for about one month to about twelve months; then further administering to the patient, in combination with (a), (b) 350 mg anti-PD-1 antibody comprising the HCVR/LCVR amino acid sequence pair of SEQ ID NOs: 1/2.

45. The method of claim 44, wherein the step of administering occurs once every 3 weeks, or once every 6 weeks.

46. A method of treating cancer or inhibiting the growth of a tumor comprising administering to a patient in need thereof:
(1) an initial loading dose comprising an anti-PD-1 antibody comprising the HCVR/LCVR amino acid sequence pair of SEQ ID NOs: 1/2; and an anti-LAG-3 antibody comprising the HCVR/LCVR amino acid sequence pair of SEQ ID NOs: 11/12; and
(2) one or more secondary doses, wherein the one or more secondary doses occur one to four weeks after the immediately preceding dose.

47. The method of claim 46, further comprising administering to a patient in need thereof:
(3) one or more tertiary doses, wherein the one or more tertiary doses occur three to twelve weeks after the immediately preceding dose.

48. The method of claim 46, wherein the one or more secondary doses occur three weeks after the immediately preceding dose.

49. The method of claim 47, wherein the one or more tertiary doses occur three weeks or six weeks after the immediately preceding dose.

50. The method of claim 46, wherein the initial loading dose comprises (a) 500 mg to 1500 mg anti-PD-1 antibody and (b) 20 or 40 mg/kg anti-LAG-3 antibody.

51. The method of claim 46, wherein the one or more secondary doses comprise: (a) 350 mg anti-PD-1 antibody and (b) 1, 3, 10, 20, or 40 mg/kg anti-LAG-3 antibody.

52. The method of claim 47, wherein the one or more tertiary doses comprise: (a) 350 mg anti-PD-1 antibody and (b) 1, 3, 10, 20, or 40 mg/kg anti-LAG-3 antibody.

53. The method of claim 46, wherein the initial loading dose comprises (a) 500 mg to 1500 mg anti-PD-1 antibody and (b) 50 mg to 8000 mg anti-LAG-3 antibody.

54. The method of claim 46, wherein the one or more secondary doses comprise: (a) 350 mg anti-PD-1 antibody and (b) 800 mg, 1000 mg, 1400 mg, 1600 mg or 2000 mg anti-LAG-3 antibody.

55. The method of claim 47, wherein the one or more tertiary doses comprise: (a) 350 mg anti-PD-1 antibody and (b) 800 mg, 1000 mg, 1400 mg, 1600 mg or 2000 mg anti-LAG-3 antibody.


SEQUENCE LISTING (20 sequences)

SEQ ID NO: 1 - protein, 117 aa, Artificial Sequence [sequence omitted]

SEQ ID NO: 2 - protein, 107 aa, Artificial Sequence [sequence omitted]

SEQ ID NO: 3 - protein, 8 aa, Artificial Sequence
  Synthetic
GFTFSNFG

SEQ ID NO: 4 - protein, 8 aa, Artificial Sequence
  Synthetic
ISGGGRDT

SEQ ID NO: 5 - protein, 10 aa, Artificial Sequence
  Synthetic
VKWGNIYFDY

SEQ ID NO: 6 - protein, 6 aa, Artificial Sequence
  Synthetic
LSINTF

SEQ ID NO: 7 - protein, 3 aa, Artificial Sequence
  Synthetic
AAS

SEQ ID NO: 8 - protein, 9 aa, Artificial Sequence
  Synthetic
QQSSNTPFT

SEQ ID NO: 9 - protein, 444 aa, Artificial Sequence
  Synthetic
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, Artificial Sequence
  Synthetic
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 123 aa, Artificial Sequence
  Synthetic
QVQLVESGGGVVQPGRSLRLSCVASGFTFSSYGMHWVRQAPGKGLEWVAIIWYDGSNKYYADSVKGRFTISRDNSKNTQYLQMNSLRAEDTAVYYCASVATSGDFDYYGMDVWGQGTTVTVSS

SEQ ID NO: 12 - protein, 107 aa, Artificial Sequence
  Synthetic
EIVLTQSPATLSLSPGERTTLSCRASQRISTYLAWYQQKPGQAPRLLIYDASKRATGIPARFSGSGSGTGFTLTISSLEPEDFAVYYCQQRSNWPLTFGGGTKVEIK

SEQ ID NO: 13 - protein, 8 aa, Artificial Sequence
  Synthetic
GFTFSSYG

SEQ ID NO: 14 - protein, 8 aa, Artificial Sequence
  Synthetic
IWYDGSNK

SEQ ID NO: 15 - protein, 16 aa, Artificial Sequence
  Synthetic
ASVATSGDFDYYGMDV

SEQ ID NO: 16 - protein, 6 aa, Artificial Sequence
  Synthetic
QRISTY

SEQ ID NO: 17 - protein, 3 aa, Artificial Sequence
  Synthetic
DAS

SEQ ID NO: 18 - protein, 9 aa, Artificial Sequence
  Synthetic
QQRSNWPLT

SEQ ID NO: 19 - protein, 449 aa, Artificial Sequence
  Synthetic
QVQLVESGGGVVQPGRSLRLSCVASGFTFSSYGMHWVRQAPGKGLEWVAIIWYDGSNKYYADSVKGRFTISRDNSKNTQYLQMNSLRAEDTAVYYCASVATSGDFDYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 214 aa, Artificial Sequence
  Synthetic
EIVLTQSPATLSLSPGERTTLSCRASQRISTYLAWYQQKPGQAPRLLIYDASKRATGIPARFSGSGSGTGFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC